(19) United States
(12) Patent Application Publication
(10) Pub. No.: US 2005/0266467 A1
(43) Pub. Date: Dec. 1, 2005

(54) Biomarkers for multiple sclerosis and methods of use thereof

(75) Inventor: Sushmita Mimi Roy, Sunnyvale, CA (US)

Correspondence Address: SHERIDAN ROSS PC, 1560 BROADWAY, SUITE 1200, DENVER, CO, 80202, US

(73) Assignee: PPD Biomarker Discovery Sciences, LLC, Wilmington, NC (US)

(21) Appl. No.: 11/134,046

(22) Filed: May 19, 2005

Related U.S. Application Data
(60) Provisional application No. 60/572,655, filed on May 19, 2004.

(51) Int. Cl. C12Q 1/68; G01N 33/53; G01N 33/567

(52) U.S. Cl. 435/6; 435/7.2

(57) ABSTRACT

Disclosed are biomarkers, the expression of which is differentially regulated in subjects with multiple sclerosis (MS) as compared to subjects that do not have MS. Also described are methods of identification of such biomarkers, and methods of using such biomarkers as targets for the development and identification of therapeutic compounds and strategies for the treatment of MS, as well as methods and kits for the diagnosis of MS.

### CROSS-REFERENCE TO RELATED APPLICATIONS

This application claims priority under 35 U.S.C. § 119(e) from U.S. Provisional Application Ser. No. 60/572,655, filed May 19, 2004. The entire disclosure of U.S. Provisional Application Ser. No. 60/572,655 is incorporated herein by reference.

### FIELD OF THE INVENTION

The present invention generally relates to the identification and use of biomarkers that are differentially expressed in patients with multiple sclerosis (MS) versus normal individuals, and methods of using such biomarkers as targets for the development of novel therapeutic strategies for the treatment of MS and in diagnostic assays.

### BACKGROUND OF THE INVENTION

Multiple sclerosis (MS) is a chronic neurological and inflammatory disease of the central nervous system (CNS). In people affected by MS, patches of damage called plaques or lesions appear in seemingly random areas of the CNS white matter. At the site of a lesion, a nerve insulating material, myelin, is lost in a process known as demyelination. Inflammation, demyelination, oligodendrocyte death, membrane damage and axonal death all contribute to the symptoms of MS. An unpredictable disease of the central nervous system, multiple sclerosis (MS) can range from relatively benign to somewhat disabling, to devastating, as communication between the brain and other parts of the body is disrupted. Many investigators believe MS to be an autoimmune disease, whereby the immune system destroys the nerve-insulating myelin. Such assaults may be linked to a yet unknown environmental trigger, such as a virus, diet, or allergy.

A physician may diagnose MS in some patients soon after the onset of the illness. In others, however, doctors may not be able to readily identify the cause of the symptoms, leading to years of uncertainty and multiple diagnoses punctuated by baffling symptoms that mysteriously wax and wane. The vast majority of patients are mildly affected, but in the worst cases, MS can render a person unable to write, speak, or walk. MS is a disease with a natural tendency to remit spontaneously, for which there is no universally effective treatment. No single laboratory test is yet available to prove or rule out MS, nor does a cure exist. Therefore, there is a great need in the art for improved diagnostic tests for MS, as well as therapeutic targets for the development of new strategies to treat MS.

### SUMMARY OF THE INVENTION

One embodiment of the present invention relates to a method to identify a compound that regulates the expression or biological activity of a polypeptide or gene encoding the polypeptide, wherein the polypeptide is differentially expressed in patients with multiple sclerosis (MS). The method includes the steps of: (a) contacting a test compound with a biomarker; and (b) identifying compounds that regulate the expression or activity of the biomarker. The biomarker is a polypeptide, a polynucleotide encoding the polypeptide, or a portion thereof, and the expression or activity of the biomarker has been associated with MS as measured by either upregulation or downregulation of the biomarker expression or activity in serum or cerebrospinal fluid from patients with MS as compared to the level of expression or activity of the biomarker in serum or cerebrospinal fluid from non-MS controls. In one aspect of this embodiment, step (b) comprises identifying compounds that: (i) increase the expression or activity of the biomarker if the expression of the biomarker is downregulated in the serum or cerebrospinal fluid of patients with MS as compared to the expression or activity of the biomarker in the serum or cerebrospinal fluid of non-MS controls; or (ii) decrease the expression or activity of the biomarker if the expression of the biomarker is upregulated in the serum or cerebrospinal fluid of patients with MS as compared to the expression or activity of the biomarker in the serum or cerebrospinal fluid of non-MS controls.

In one aspect, the biomarker is a polynucleotide expressed by a test cell, and step (b) comprises identifying compounds that regulate the expression of the polynucleotide in the presence of the test compound as compared to in the absence of the test compound. Expression of the polynucleotide can be measured, for example, by measuring transcription of the polynucleotide or translation of a protein encoded by the polynucleotide. In another aspect, the biomarker is a polypeptide, and step (b) comprises identifying compounds that regulate the activity of the polypeptide in the presence of the test compound as compared to in the absence of the test compound.

In one aspect, the biomarker is a polypeptide or a fragment of a polypeptide that is identified by liquid chromatography mass spectrophotometry as being differentially expressed in serum or cerebrospinal fluid from a subject with MS as compared to a subject that does not have MS. Such a biomarker can include, for example, a polypeptide or biologically active fragment thereof comprising an amino acid sequence selected from: SEQ ID NO:1-26. In another aspect, such a biomarker can include a polypeptide or biologically active fragment thereof selected from: antithrombin III; α-2 glycoprotein 1, zinc; transthyretin (prealbumin); NADH dehydrogenase (ubiquinone) 1 beta subcomplex, 2; neurotrimin; orosomucoid 1 precursor (α-1-acid glycoprotein-1); leucine-rich α-2-glycoprotein; leucine-rich repeat protein; and α-1-antitrypsin. In another aspect, such a biomarker can include a polypeptide or a biologically active fragment thereof comprising a polypeptide having a mass-to-charge value and a retention time (RT) value within 10% of the mass-to-charge value and RT value of a biomarker component selected from: component 3991, component 2298, component 100, component 132, component 4355, component 136, component 153, component 4024, component 3935, component 240, component 261, component 319, component 3573, component 359, component 373, component 376, component 377, component 408, component 573, component 579, component 2989, component 3110, component 4098, component 634, component 658, component 747, component 787, component 874, component 880, component 982, component 4164, component 4168, component 1041, component 4170, component 1104, component 4190, component 1166, component 1215, component 2607, component 3237, component 1329, component 1333, component 2844, component 1453, component 1519, component 1529, component 1546, component 2440, component 1687, component 1808, component 4797, component 1862, component 1895, component 2058, component 2970, and component 2259. These components are described in detail in Table 2B.

Yet another embodiment of the invention relates to a method to diagnose multiple sclerosis (MS). The method includes the steps of: (a) detecting in a sample of serum or cerebrospinal fluid from a patient to be tested the level of expression of at least one biomarker chosen from a panel of biomarkers whose expression has been associated with MS as measured by either upregulation or downregulation of biomarker expression in serum or cerebrospinal fluid from patients with MS as compared to the level of expression of the biomarkers in serum or cerebrospinal fluid from non-MS controls; (b) comparing the level of expression of the biomarker or biomarkers detected in the patient sample to a level of expression of the biomarker or biomarkers that has been associated with MS and a level of expression of the biomarker or biomarkers that has been associated with non-MS controls; and (c) diagnosing MS in the patient if the expression level of the biomarker or biomarkers in the patient sample is statistically more similar to the expression level of the biomarker or biomarkers that has been associated with MS than the expression level of the biomarker or biomarkers that has been associated with the non-MS controls. The biomarkers or panel of biomarkers can include any of the biomarkers described above. Any number of biomarkers can be detected, including, but not limited to, 2, 5, 10 or more biomarkers. In one aspect, the level of expression of the biomarker that has been associated with MS and the level of expression of the biomarker that has been associated with non-MS controls has been predetermined.

In one aspect of this embodiment, step (a) comprises detecting in the patient sample the expression of at least one polypeptide, biologically active fragment thereof, or polynucleotide encoding the polypeptide or biologically active fragment thereof, wherein the polypeptide comprises an amino acid sequence selected from the group consisting of SEQ ID NO: 1-26; step (b) comprises comparing the level of expression of the polypeptide, fragment thereof or polynucleotide detected in the patient sample to a level of expression of the polypeptide, fragment thereof or polynucleotide that has been associated with MS and to a level of expression of the polypeptide, fragment thereof or polynucleotide that has been associated with non-MS controls; and step (c) comprises diagnosing MS in the patient, if the expression of the polypeptide, fragment thereof or polynucleotide in the patient sample is statistically more similar to the expression level of the polypeptide, fragment thereof or polynucleotide that has been associated with MS than with non-MS controls.

In one aspect of this embodiment, expression of the biomarker is detected by measuring amounts of transcripts of a gene encoding a polypeptide biomarker in the patient serum or cerebrospinal fluid. In another aspect, expression of the biomarker is detected by detecting the expression of a protein. In another aspect, the biomarker is a polypeptide or a fragment thereof, and wherein the biological activity of the polypeptide is detected. In another aspect, the biomarker is a polypeptide, and wherein the expression of the polypeptide is detected using an antibody that selectively binds to the polypeptide, or an antigen binding fragment thereof.

An additional aspect of this embodiment can include determining if the patient has relapsing/remitting MS or a progressive form of MS. The step of determining comprises: (a) detecting in the sample the level of expression of at least one biomarker chosen from a panel of biomarkers whose expression has been associated with relapsing/remitting MS and or a progressive form of MS as measured by either upregulation or downregulation of biomarker expression in serum or cerebrospinal fluid from patients with relapsing/remitting MS as compared to the level of expression of the biomarkers in serum or cerebrospinal fluid from subjects with a progressive form of MS; (b) comparing the level of expression of the biomarker detected in the patient sample to a level of expression of the biomarker that has been associated with the relapsing/remitting MS and to a level of expression of the biomarker that has been associated with the progressive form of MS; and (c) diagnosing relapsing/remitting MS in the patient, if the expression of the biomarker in the patient sample is statistically more similar to the expression level of the biomarker that has been associated with relapsing/remitting MS than with the progressive form of MS, or diagnosing progressive form of MS in the patient, if the expression of the biomarker in the patient sample is statistically more similar to the expression level of the biomarker that has been associated with progressive form of MS than with relapsing/remitting MS.

Another embodiment of the invention relates to a plurality of antibodies or antigen binding fragments thereof for the detection of the expression of proteins that are associated with multiple sclerosis (MS) in a patient. The plurality of antibodies or antigen binding fragments thereof consists of at least two antibodies or antigen binding fragments thereof, each of which selectively binds to a polypeptide, the expression of which is regulated differently in serum or cerebrospinal fluid of patients with MS as compared to serum or cerebrospinal fluid of individuals that do not have MS. In one aspect, each antibody or antigen binding fragment thereof selectively binds to a polypeptide comprising an amino acid sequence selected from the group consisting of SEQ ID NOs:1-26. In another aspect, the plurality comprises antibodies or antigen binding fragments thereof that selectively bind to at least two polypeptides comprising an amino acid sequence selected from the group consisting of SEQ ID NOs:1-26. In another aspect, each antibody or antigen binding fragment thereof selectively binds to a polypeptide selected from the group of: antithrombin III; α-2 glycoprotein 1, zinc; transthyretin (prealbumin); NADH dehydrogenase (ubiquinone) 1 beta subcomplex, 2; neurotrimin; orosomucoid 1 precursor (α-1-acid glycoprotein-1); leucine-rich α-2-glycoprotein; leucine-rich repeat protein; and α-1-antitrypsin. In yet another aspect, each antibody or antigen binding fragment thereof selectively binds to a polypeptide comprising a polypeptide having a mass-to-charge value and a retention time (RT) value within 10% of the mass-to-charge value and RT value of a biomarker component selected from: component 3991, component 2298, component 100, component 132, component 4355, component 136, component 153, component 4024, component 3935, component 240, component 261, component 319, component 3573, component 359, component 373, component 376, component 377, component 408, component 573, component 579, component 2989, component 3110, component 4098, component 634, component 658, component 747, component 787, component 874, component 880, component 982, component 4164, component 4168, component 1041, component 4170, component 1104, component 4190, component 1166, component 1215, component 2607, component 3237, component 1329, component 1333, component 2844, component 1453, component 1519, component 1529, component 1546, component 2440, component 1687, component 1808, component 4797, component 1862, component 1895, component 2058, component 2970, and component 2259. In another aspect, the antibodies or antigen binding fragments thereof are immobilized on a substrate. In yet another aspect, the antibodies or antigen binding fragments thereof are conjugated to detectable markers.

### BRIEF DESCRIPTION OF THE DRAWINGS OF THE INVENTION

FIG. 1 is a schematic diagram showing cerebrospinal fluid (CSF) sample processing and LC-MS profiling.

FIG. 2 is a graph showing the coefficients of variation (CV) distribution among 20 individuals, for 4000 ions measured directly in the CSF proteome.

FIG. 3 is a graph showing proteins discovered in CSF that are up- or down-regulated significantly in relapsing-remitting multiple sclerosis.

FIG. 4 is a graph showing proteins discovered in serum that are up- or down-regulated significantly in relapsing-remitting multiple sclerosis.

### DETAILED DESCRIPTION OF THE INVENTION

The present invention generally relates to a method for differential protein expression profiling in multiple sclerosis (MS) using a direct LC-MS approach for quantitation. The method has been developed for cerebrospinal fluid analysis, as well as serum analysis. In addition, the present invention relates to the identification and quantification of novel biomarkers for MS and the use of these biomarkers as diagnostic tools and as targets for novel therapeutic strategies for the treatment of MS.

The method described herein is a liquid chromatography-mass spectrometry (LC-MS)-based, sensitive method for differential quantification and identification of CSF proteins. Spectral interpretation and intensity-normalization software (MassView™ software, version 2.0; SurroMed) was used to quantify the difference in expression level of proteins between controls and MS patients in CSF and serum, using relative component intensities without the use of isotope tagging. By comparing CSF and serum profiles in relapsing-remitting MS patients and unaffected controls, an application of biomarker discovery has been demonstrated.

The method of the present invention may also be used to identify additional biomarkers useful as targets or in the diagnosis of MS and particularly, biomarkers that discriminate patients with relapsing/remitting MS from the three progressive forms of the disease.

According to the present invention, the term “multiple sclerosis” (MS) can be used to describe the art-recognized disease characterized by inflammation, demyelination, oligodendrocyte death, membrane damage and axonal death. MS can be more particularly categorized as either relapsing/remitting MS (observed in 85-90% of patients) or progressive MS. In some embodiments, MS can be characterized as one of four main varieties as defined in an international survey of neurologists (Lublin and Reingold, 1996, Neurology 46(4):907-11), which are namely, relapsing/remitting MS, secondary progressive MS, progressive/relapsing MS, or primary progressive MS (PPMS). Relapsing/remitting MS is characterized by relapses during which time new symptoms can appear and old ones resurface or worsen. The relapses are followed by periods of remission, during which time the person fully or partially recovers from the deficits acquired during the relapse. After a number of years, many people who have had relapsing/remitting MS will pass into a secondary, progressive phase of the disease, known as secondary progressive MS. Secondary progressive MS is characterized by a gradual worsening of the disease between relapses, which ultimately these merge into a general progression. Progressive/relapsing MS follows a progressive course from onset, punctuated by relapses. There is significant recovery immediately following a relapse but between relapses there is a gradual worsening of symptoms. Finally, primary progressive multiple sclerosis (PPMS) is characterized by a gradual progression of the disease from its onset with no remissions at all. There may be periods of a leveling off of disease activity and, as with secondary progressive, there may be good and bad days or weeks. PPMS often migrates into the brain, but is less likely to damage brain areas than relapsing/remitting or secondary progressive and thereby cause cognitive problems.

Currently, diagnosis of MS is difficult and a patient may face years of misdiagnosis or non-diagnosis of a condition punctuated by baffling symptoms that mysteriously wax and wane. The ability to aid in the diagnosis of MS using a simple test would be of significant benefit in the management of the disease. Such a test would facilitate the earlier diagnosis of this disease, when treatment might be more effective. It would also decrease the need for a number of expensive and invasive studies used to exclude secondary causes of various symptoms of the disease. In addition, there would be value in the development of a diagnostic test that could discriminate among the various forms of MS. The present invention provides a solution through a novel method to diagnose a patient with MS, including a method that can discriminate among the various forms of MS.

In addition, there is no cure for MS and current therapeutic strategies are, at best, useful for reducing some symptoms or complications of the disease, but many can have adverse and/or systemic side effects that make their use less than desirable. For example, current therapy for MS includes cytokine therapy (e.g., interferon beta), corticosteroid therapy, immunotherapy (e.g., monoclonal antibodies specific for lymphocytes), ion channel blockers, and anti-viral therapies, along with a battery of therapies for various complications of the disease, including pain, depression, fatigue, urinary dysfunction, bowel dysfunction, sexual dysfunction, muscle spasticity and tremors, and nausea. Moreover, given the multiple forms of MS and continued debate in the art regarding how MS develops and progresses, there is a great need in the art for new targets for drug development and improved therapeutic strategies for the treatment of MS. The present invention identifies multiple biomarkers that are associated with MS, which can now be studied in more detail and/or be used as targets for the discovery of other modulators of disease or therapeutic agents for the treatment of MS. Moreover, the present invention can be used to monitor progression of a disease and/or the efficacy of disease treatments.

As used herein, the terms “patient”, “subject”, “a subject who has MS”, “a patient who has MS”, “an MS subject”, “an MS patient”, and similar phrases, are intended to refer to subjects who have been diagnosed with MS. The terms “non-MS control”, “non-subject”, “a subject who does not have MS”, “a patient who does not have MS”, “normal control” or “an individual who does not have MS”, and similar phrases, are intended to refer to a subject who has not been diagnosed with MS. A non-MS control may be healthy and have no other disease, or such an individual may have a disease other than MS.

As used herein, the term “biological sample” includes a sample of any cell type or from any tissue or body fluid, body fluids including, but not limited to: cerebrospinal fluid (CSF), serum, plasma, blood, urine, prostatic fluid, saliva or fluid from any suitable tissue. In a preferred embodiment, the biological sample is a CSF sample.

The data set forth in Tables 2A and 2B reflect the method used to identify the markers. When a sample is processed and analyzed as described in the Example, the retention time of the marker is about the value stated for the marker (e.g., within about 10% of the value stated, within about 5% of the value stated, within about 1% of the value stated, or any percentage between about 10% and about 1%, in whole percentage increments) and has a mass to charge ratio of about the value stated for the marker (within about 10% of the value stated, within about 5% of the value stated, within about 1% of the value stated, or any percentage between about 10% and about 1%, in whole percentage increments).

A profile of individual biomarkers to use in a method of the invention, including a matrix of two or more markers, can be generated using the LC-MS technique described in detail in the Example.

Biomarkers

The invention is based in part on the discovery that certain polypeptides and metabolites are differentially expressed in CSF samples obtained from subjects diagnosed with MS as compared to CSF samples obtained from control subjects without MS. Tables 2A and 2B list biomarkers (also referred to herein as “components”) of the invention that were found at significantly different levels in CSF samples obtained from subjects with MS as compared with control subjects without MS (p<0.05). Accordingly, one embodiment of the present invention relates to biomarkers that are differentially expressed in patients with MS as compared to individuals that do not have MS, such biomarkers being useful in diagnostic assays described herein, and as targets for therapeutic drug or treatment design.

The term “biomarker” or “marker”, as used herein, can refer to polypeptide or metabolite described herein or to a polynucleotide (including a gene) that encodes a polypeptide identified by the invention. In addition, the term “biomarker” can be generally used to refer to any portion of such a polypeptide or polynucleotide that can identify or correlate with the full-length polypeptide or polynucleotide, for example, in an assay of the invention. Biomarkers also include any precursors and successors of polypeptides and polynucleotides of the invention, as well as polypeptides and polynucleotides substantially homologous to polypeptides and polynucleotides of the invention. Accordingly, a biomarker useful in the present invention is any polynucleotide, polypeptide or metabolite, the expression of which is regulated (up or down) in a patient with a condition (e.g., MS) as compared to a normal control. Selected sets of one, two, three, and more preferably several more of the biomarkers of this invention (up to the number equivalent to all of the biomarkers, including any intervening number, in whole number increments, e.g., 1, 2, 3, 4, 5, 6 . . . ) can be used as end-points for rapid diagnostics or prognostics for MS, and/or as targets for the development of therapeutic drugs and strategies for the treatment of MS. In one embodiment, larger numbers of the biomarkers identified herein are used in a diagnostic assay of the invention (e.g., at least 10 genes or more), since the accuracy of the assay improves as the number of biomarkers screened increases.

Of the several thousand molecular ions quantified and several hundred proteins that were profiled, the present inventors have identified multiple proteins, the expression of which are regulated differentially in patients with MS as compared to subjects without MS. More particularly, the proteins can be grouped into the following main categories: (1) proteins that are selectively (i.e., exclusively or uniquely) upregulated in the serum or cerebrospinal fluid (CSF) of patients with MS as compared to normal controls (Tables 2A and 2B); and (2) proteins that are selectively downregulated in the serum or CSF of patients with MS as compared to normal controls (Tables 2A and 2B).

Table 2A shows the proteome data identifying 40 biomarkers of unavailable name that were determined by the present inventor to be significantly (at p<0.05) regulated (up or down) in the CSF of patients with MS as compared to normal controls, sorted in ascending order of the mass to charge ratio of the ion (m/z). Each biomarker polypeptide is identified in Table 2A by the mass to charge ratio (m/z); chromatographic retention time (RT); the charge state of a molecular ion (z); protonated parent mass (M+H); expression ratio (exp. ratio), which is a ratio of mean group intensities indicating the relative normalized signal for disease group compared to control; fold change (an expression change factor where positive indicates an intensity increase and negative indicates a decrease versus the control); the trend toward up- or downregulation; and the p-value of the univariate test.

Table 2B shows the proteome data identifying 26 biomarkers of available name that were determined by the present inventor to be significantly (at p<0.05) regulated (up or down) in the CSF of patients with MS as compared to normal controls. Each biomarker polypeptide is identified in Table 2B by the mass to charge ratio (m/z); chromatographic retention time (RT); the charge state of a molecular ion (z); protonated parent mass (M+H); identification number from the National Center for Biotechnology Information (NCBI) sequence database (Accession # and gi #); additional information such as the name and/or sequence of the peptide marker as contained in the NCBI queried database and database searching using the TurboQUEST program; expression ratio (exp. ratio); fold change (an expression change factor where positive indicates an intensity increase and negative indicates a decrease versus the control); the trend toward up- or downregulation; and the p-value of the univariate test.

Many of the polypeptides listed in Tables 2A and 2B are fragments of complete proteins (“parent proteins”), either because they were present as fragments in the sample or as a result of the trypsin digestion that was performed during the processing of certain fractions of the sample. The parent proteins are included as polypeptide markers. In many cases, the sequence of the parent protein can be ascertained from the amino acid sequence of the fragment by searching a protein sequence database. Accordingly, polypeptide biomarkers of the invention include, but are not limited to: antithrombin III; α-2 glycoprotein 1, zinc; transthyretin (prealbumin); NADH dehydrogenase (ubiquinone) 1 beta subcomplex, 2; neurotrimin; orosomucoid 1 precursor (α-1-acid glycoprotein-1); leucine-rich α-2-glycoprotein; leucine-rich repeat protein; and α-1-antitrypsin, in addition to fragments and homologues of any of such polypeptides.

In another preferred embodiment, biomarkers include any polypeptide or a biologically active fragment thereof comprising a polypeptide having a mass-to-charge value and a retention time (RT) value within 10%, particularly within 5%, more particularly within 1%. and (ii) an RT value within 10%, particularly within 5%, more particularly within 1% (or any percentage between about 10% and about 1%, in whole percentage increments), of the mass-to-charge value and RT value of a biomarker component listed in Table 2B. The components in Table 2B are identified by a component number that corresponds to a specific mass-to-charge value and a specific RT value that identifies the component. The components therefore, include: component 3991, component 2298, component 100, component 132, component 4355, component 136, component 153, component 4024, component 3935, component 240, component 261, component 319, component 3573, component 359, component 373, component 376, component 377, component 408, component 573, component 579, component 2989, component 3110, component 4098, component 634, component 658, component 747, component 787, component 874, component 880, component 982, component 4164, component 4168, component 1041, component 4170, component 1104, component 4190, component 1166, component 1215, component 2607, component 3237, component 1329, component 1333, component 2844, component 1453, component 1519, component 1529, component 1546, component 2440, component 1687, component 1808, component 4797, component 1862, component 1895, component 2058, component 2970, or component 2259.

As one of skill in the art will appreciate, the physical and chemical properties presented in the Tables are sufficient to distinguish the component from other materials. In particular, the components are uniquely identified by the mass to charge ratio (m/z) and the retention time (RT).

Some variation is inherent in the measurements of the physical and chemical characteristics of the markers. The magnitude of the variation depends to some extent on the reproducibility of the separation means and the specificity and sensitivity of the detection means used to make the measurement. Preferably, the method and technique used to measure the markers is sensitive and reproducible.

The retention time and mass to charge ratio may vary to some extent depending on a number of factors relating to the protocol used for the chromatography and the mass spectrometry parameters such as the solvent composition or flow rate. Preferably, sample preparation and analysis conditions are carefully controlled. One of skill in the art, however, will appreciate that the possibility of contamination or measurement of artifacts can never be completely eliminated.

The data set forth in Tables 2A and 2B reflect the method used to identify the markers. When a sample is processed and analyzed as described in the Example, the retention time of the marker is about the value stated for the marker (e.g., within about 10% of the value stated, within about 5% of the value stated, within about 1% of the value stated, or any percentage between about 10% and about 1%, in whole percentage increments) and has a mass to charge ratio of about the value stated for the marker (within about 10% of the value stated, within about 5% of the value stated, within about 1% of the value stated, or any percentage between about 10% and about 1%, in whole percentage increments).

Polypeptide Biomarkers

In one embodiment, the invention provides a biomarker polypeptide or metabolite having (i) a mass-to-charge value within 10%, particularly within 5%, more particularly within 1%. and (ii) an RT value within 10%, particularly within 5%, more particularly within 1% (or any percentage between about 10% and about 1%, in whole percentage increments), of the m/z and RT values stated, respectively, for a polypeptide described in Table 2A or 2B, or for any other polypeptide that can be identified as differentially expressed in patients with MS using the identification method of the invention. Also included in the invention is a polypeptides that is a fragment, precursor, successor or modified version of a marker described in Table 2A or Table 2B. Preferred polypeptide biomarkers include, but are not limited to: antithrombin III; α-2 glycoprotein 1, zinc; transthyretin (prealbumin); NADH dehydrogenase (ubiquinone) 1 beta subcomplex, 2; neurotrimin; orosomucoid 1 precursor (α-1-acid glycoprotein-1); leucine-rich α-2-glycoprotein; leucine-rich repeat protein; and α-1-antitrypsin, or any fragment or homologue thereof, including any biologically active fragment or homologue thereof.

In another embodiment, the invention provides a polypeptide or a biologically active fragment thereof comprising a polypeptide having a mass-to-charge value and a retention time (RT) value within 10%, particularly within 5%, more particularly within 1%. and (ii) an RT value within 10%, particularly within 5%, more particularly within 1% (or any percentage between about 10% and about 1%, in whole percentage increments), of the mass-to-charge value and RT value of a biomarker component listed in Table 2B, as discussed above. The components therefore, include: component 3991, component 2298, component 100, component 132, component 4355, component 136, component 153, component 4024, component 3935, component 240, component 261, component 319, component 3573, component 359, component 373, component 376, component 377, component 408, component 573, component 579, component 2989, component 3110, component 4098, component 634, component 658, component 747, component 787, component 874, component 880, component 982, component 4164, component 4168, component 1041, component 4170, component 1104, component 4190, component 1166, component 1215, component 2607, component 3237, component 1329, component 1333, component 2844, component 1453, component 1519, component 1529, component 1546, component 2440, component 1687, component 1808, component 4797, component 1862, component 1895, component 2058, component 2970, or component 2259.

As used herein, the term “polypeptide” refers to a polymer of amino acid residues, and includes a sufficient number of amino acids to identify the polypeptide as a biomarker in the present invention. Therefore, a polypeptide can include a peptide, an oligopeptide, a protein, and may be composed of two or more polypeptide chains. A polypeptide can be linear or branched. A polypeptide can comprise modified amino acid residues, amino acid analogs or non-naturally occurring amino acid residues and can be interrupted by non-amino acid residues. Included within the definition are amino acid polymers that have been modified, whether naturally or by intervention, such as formation of a disulfide bond, glycosylation, lipidation, methylation, acetylation, phosphorylation, or by manipulation, such as conjugation with a labeling component.

As used herein, the term “homologue” is used to refer to a polypeptide which differs from a naturally occurring polypeptide by one or more minor modifications or mutations to the naturally occurring polypeptide, but which maintains the overall basic protein and side chain structure of the naturally occurring form (i.e., such that the homologue is identifiable as being related to the wild-type polypeptide). Such changes include, but are not limited to: changes in one or a few amino acid side chains; changes one or a few amino acids, including deletions (e.g., a truncated version of the protein or peptide) insertions and/or substitutions; changes in stereochemistry of one or a few atoms; and/or minor derivatizations, including but not limited to: methylation, farnesylation, geranyl geranylation, glycosylation, carboxymethylation, phosphorylation, acetylation, myristoylation, prenylation, palmitation, and/or amidation. A homologue can have either enhanced, decreased, or substantially similar properties as compared to the naturally occurring polypeptide. Homologues can include synthetically produced homologues, naturally occurring allelic variants of a given protein or domain, or homologous sequences from organisms other than the organism from which the reference polypeptide was derived.

As used herein, in one embodiment, two polypeptides are “substantially homologous” or “homologues” when there is at least 70% homology, at least 80% homology, at least 90% homology, at least 95% homology or at least 99% homology between their amino acid sequences, or when polynucleotides encoding the polypeptides are capable of forming a stable duplex with each other. As used herein, unless otherwise specified, reference to a percent (%) identity refers to an evaluation of homology which is performed using: (1) a BLAST 2.0 Basic BLAST homology search using blastp for amino acid searches, blastn for nucleic acid searches, and blastX for nucleic acid searches and searches of translated amino acids in all 6 open reading frames, all with standard default parameters, wherein the query sequence is filtered for low complexity regions by default (described in Altschul, S. F., Madden, T. L., Schääffer, A. A., Zhang, J., Zhang, Z., Miller, W. & Lipman, D. J. (1997) “Gapped BLAST and PSI-BLAST: a new generation of protein database search programs.” Nucleic Acids Res. 25:3389, incorporated herein by reference in its entirety); (2) a BLAST 2 alignment (using the parameters described below); (3) and/or PSI-BLAST with the standard default parameters (Position-Specific Iterated BLAST). It is noted that due to some differences in the standard parameters between BLAST 2.0 Basic BLAST and BLAST 2, two specific sequences might be recognized as having significant homology using the BLAST 2 program, whereas a search performed in BLAST 2.0 Basic BLAST using one of the sequences as the query sequence may not identify the second sequence in the top matches. In addition, PSI-BLAST provides an automated, easy-to-use version of a “profile” search, which is a sensitive way to look for sequence homologues. The program first performs a gapped BLAST database search. The PSI-BLAST program uses the information from any significant alignments returned to construct a position-specific score matrix, which replaces the query sequence for the next round of database searching. Therefore, it is to be understood that percent identity can be determined by using any one of these programs.

As used herein, a “fragment” of a polypeptide refers to a single or a plurality of amino acid residues comprising an amino acid sequence that has at least 5 contiguous amino acid residues, at least 10 contiguous amino acid residues, at least 20 contiguous amino acid residues, at least 30 contiguous amino acid residues, at least 40 contiguous amino acid residues, at least 50 contiguous amino acid residues, at least 60 contiguous amino acid residues, at least 70 contiguous amino acid residues, at least 80 contiguous amino acid residues, at least 90 contiguous amino acid residues, or at least 100 contiguous amino acid residues of a sequence of the polypeptide, or any number of residues between 5 and 100, in whole number increments.

As used herein, a polypeptide is referred to as “isolated” when it has been removed from its natural milieu (i.e., that has been subject to human manipulation), and can include purified polypeptides, partially purified polypeptides, synthetically produced polypeptides, and recombinantly produced polypeptides, for example. As such, “isolated” does not reflect the extent to which the polypeptide has been purified.

In some embodiments, a biomarker of the invention is a member of a biological pathway. As used herein, the term “precursor” or “successor” refers to molecules that precede or follow the biomarker. Thus, once a biomarker is identified as a member of one or more biological pathways, the present invention can include additional members of the biological pathway that come before (are upstream of or a precursor of) or follow (are downstream of) the biomarker. Such identification of biological pathways and their members is within the skill of one in the art.

Polypeptide and metabolite markers may be isolated by any suitable method known in the art. Native polypeptide and metabolite markers can be purified from natural sources by standard methods known in the art such as chromatography, centrifugation, differential solubility or immunoassay. In one embodiment, polypeptide and metabolite markers may be isolated from a serum sample using, for example, the chromatographic methods disclosed herein or affinity purification using substrate-bound antibodies that specifically bind to the marker. Metabolite makers may be synthesized using the techniques of organic and inorganic chemistry. Given the amino acid sequence or the corresponding DNA, cDNA, or mRNA that encodes them, polypeptides markers may be synthesized using recombinant or chemical methods. For example, polypeptide markers can be produced by transforming a host cell with a nucleotide sequence encoding the polypeptide marker and cultured under conditions suitable for expression and recovery of the encoded protein from the cell culture. (See, e.g., Hunkapiller et al., Nature 310:105-111, 1984).

Polynucleotide Biomarkers

The present invention also includes polynucleotides that encode any of the polypeptides identified by the biomarker identification method of the invention and/or as described above and in Tables 2A or 2B or that encode any other polypeptide that can be identified as differentially expressed in patients with MS using the identification method of the invention, or that encode a molecule that comprises such a polypeptide or a polypeptide having substantial homology with a component set forth in Tables 2A or 2B.

In accordance with the present invention, an isolated polynucleotide, or an isolated nucleic acid molecule, is a nucleic acid molecule that has been removed from its natural milieu (i.e., that has been subject to human manipulation), its natural milieu being the genome or chromosome in which the nucleic acid molecule is found in nature. As such, “isolated” does not necessarily reflect the extent to which the polynucleotide has been purified, but indicates that the molecule does not include an entire genome or an entire chromosome in which the nucleic acid molecule is found in nature. Polynucleotides useful in the present invention include a portion of a gene (sense or non-sense strand) that is suitable for use as a hybridization probe or PCR primer for the identification of a full-length gene (or portion thereof) in a given sample (e.g., a CSF or serum sample), a gene, or any portion of a gene, as well as a reporter gene.

The minimum size of a polynucleotide of the present invention is a size sufficient to encode a polypeptide having a desired biological activity, sufficient to form a probe or oligonucleotide primer that is capable of forming a stable hybrid with the complementary sequence of a polynucleotide encoding the natural polypeptide, or to otherwise be used as a target in an assay, in a diagnostic assay, or in any therapeutic method discussed herein. The minimum size of a polynucleotide that is used as an oligonucleotide probe or primer is at least about 5 nucleotides in length, and preferably ranges from about 5 to about 50 or about 500 nucleotides or greater (1000, 2000, etc.), including any length in between, in whole number increments (i.e., 5, 6, 7, 8, 9, 10, . . . 33, 34, . . . 256, 257, . . . 500 . . . 1000 . . . ). “Hybridization” has the meaning that is well known in the art, that is, the formation of a duplex structure by two single-stranded nucleic acids due to complementary base pairing.

In one embodiment of the present invention, any amino acid sequence described herein can be produced with from at least one, and up to about 20, additional heterologous amino acids flanking each of the C- and/or N-terminal ends of the specified amino acid sequence. The resulting protein or polypeptide can be referred to as “consisting essentially of” the specified amino acid sequence. According to the present invention, the heterologous amino acids are a sequence of amino acids that are not naturally found (i.e., not found in nature, in vivo) flanking the specified amino acid sequence, or that are not related to the function of the specified amino acid sequence, or that would not be encoded by the nucleotides that flank the naturally occurring nucleic acid sequence encoding the specified amino acid sequence as it occurs in the gene, if such nucleotides in the naturally occurring sequence were translated using standard codon usage for the organism from which the given amino acid sequence is derived. Similarly, the phrase “consisting essentially of”, when used with reference to a nucleic acid sequence herein, refers to a nucleic acid sequence encoding a specified amino acid sequence that can be flanked by from at least one, and up to as many as about 60, additional heterologous nucleotides at each of the 5′ and/or the 3′ end of the nucleic acid sequence encoding the specified amino acid sequence. The heterologous nucleotides are not naturally found (i.e., not found in nature, in vivo) flanking the nucleic acid sequence encoding the specified amino acid sequence as it occurs in the natural gene or do not encode a protein that imparts any additional function to the protein or changes the function of the protein having the specified amino acid sequence.

One embodiment of the invention relates to a plurality of polynucleotides for the detection of the expression of biomarkers that are differentially regulated in serum or CSF of patients with MS. The plurality of polynucleotides consists of, or consists essentially of, at least two polynucleotide probes that are complementary to RNA transcripts, or nucleotides derived therefrom, of at least one polynucleotide, the polypeptide encoded by which has been identified herein as being differentially regulated in the serum or CSF of patients with MS. As such, the plurality of polynucleotides is distinguished from previously known nucleic acid arrays and primer sets. The plurality of polynucleotides within the above-limitation includes at least two or more polynucleotide probes (e.g., at least 2, 3, 4, 5, 6, and so on, in whole integer increments, up to all of the possible probes) that are complementary to RNA transcripts, or nucleotides derived therefrom, of at least one polynucleotide, and preferably, at least 2 or more polynucleotides, encoding polypeptides identified by the present invention. Such polynucleotides are selected from any of the polynucleotides encoding a polypeptide listed in the tables provided herein and can include any number of polynucleotides, in whole integers (e.g., 1, 2, 3, 4, . . . ) up to all of the polynucleotides represented by a biomarker described herein, or that can be identified in MS patients using the methods described herein. Multiple probes can also be used to detect the same polynucleotide or to detect different splice variants of the same gene. In one aspect, each of the polynucleotides in the plurality is at least 5 nucleotides in length.

Antibodies and Binding Partners

The invention also includes antibodies, or antigen binding fragments thereof, that specifically bind to a polypeptide marker, a metabolite marker or a polynucleotide marker, in particular that bind to a component described in Tables 2A or 2B or any other component that can be identified as differentially expressed in patients with MS using the identification method of the invention. The invention also provides antibodies that specifically bind to a polypeptide having substantial homology with a component set forth in Tables 2A or 2B.

The invention provides antibodies, or antigen binding fragments thereof, that specifically bind to a polypeptide or metabolite of the invention having (i) a mass-to-charge value and (ii) an RT value of about the values stated, respectively, for a marker described in Tables 2A or 2B. In particular, the invention provides antibodies that specifically bind to a polypeptide or metabolite of the invention having (i) a mass-to-charge value within 10%, particularly within 5%, more particularly within 1%, or any percentage between about 10% and about 1%, in whole percentage increments, and (ii) an RT value within 10%, particularly within 5%, more particularly within 1%, or any percentage between about 10% and about 1%, in whole percentage increments, of the m/z and RT values stated, respectively, for a component described in Table 2A or 2B or any other component that can be identified as differentially expressed in patients with MS using the identification method of the invention.

In another embodiment, the invention provides antibodies, or antigen binding fragments thereof, that specifically bind to a component that is a fragment, modification, precursor or successor of a polypeptide or metabolite of the invention described in Tables 2A or 2B.

In one embodiment, the present invention provides a plurality of antibodies, or antigen binding fragments thereof, for the detection of biomarkers, the expression of which is differentially regulated in the serum or CSF of patients with MS. In addition, the plurality of antibodies, or antigen binding fragments thereof, comprises antibodies, or antigen binding fragments thereof, that selectively bind to a biomarker provided herein.

According to the present invention, a plurality of antibodies, or antigen binding fragments thereof, refers to at least 2, and more preferably at least 3, and more preferably at least 4, and more preferably at least 5, and more preferably at least 6, and more preferably at least 7, and more preferably at least 8, and more preferably at least 9, and more preferably at least 10, and so on, in increments of one, up to any suitable number of antibodies, or antigen binding fragments thereof, including antibodies representing all of the biomarkers described herein.

According to the present invention, the phrase “selectively binds to” refers to the ability of an antibody or antigen binding fragment thereof to preferentially bind to specified proteins. More specifically, the phrase “selectively binds” refers to the specific binding of one protein to another (e.g., an antibody or antigen binding fragment thereof to an antigen), wherein the level of binding, as measured by any standard assay (e.g., an immunoassay), is statistically significantly higher than the background control for the assay. For example, when performing an immunoassay, controls typically include a reaction well/tube that contain antibody or antigen binding fragment alone (i.e., in the absence of antigen), wherein an amount of reactivity (e.g., non-specific binding to the well) by the antibody or antigen binding fragment thereof in the absence of the antigen is considered to be background. Binding can be measured using a variety of methods standard in the art including enzyme immunoassays (e.g., ELISA), immunoblot assays, etc.).

As used herein, the term “specifically binding,” refers to the interaction between binding pairs such as an antibody and an antigen with an affinity constant of at most 10⁻⁶ moles/liter, at most 10⁻⁷ moles/liter, or at most 10⁻⁸ moles/liter.

Limited digestion of an immunoglobulin with a protease may produce two fragments. An antigen binding fragment is referred to as an Fab, an Fab′, or an F(ab′)₂ fragment. A fragment lacking the ability to bind to antigen is referred to as an Fc fragment. An Fab fragment comprises one arm of an immunoglobulin molecule containing a L chain (V_{L}+C_{L} domains) paired with the VH region and a portion of the CH region (CH1 domain). An Fab′ fragment corresponds to an Fab fragment with part of the hinge region attached to the CH1 domain. An F(ab′)₂ fragment corresponds to two Fab′ fragments that are normally covalently linked to each other through a di-sulfide bond, typically in the hinge regions.

Isolated antibodies of the present invention can include serum containing such antibodies, or antibodies that have been purified to varying degrees. Whole antibodies of the present invention can be polyclonal or monoclonal. Alternatively, functional equivalents of whole antibodies, such as antigen binding fragments in which one or more antibody domains are truncated or absent (e.g., Fv, Fab, Fab′, or F(ab)₂ fragments), as well as genetically-engineered antibodies or antigen binding fragments thereof, including single chain antibodies or antibodies that can bind to more than one epitope (e.g., bi-specific antibodies), or antibodies that can bind to one or more different antigens (e.g., bi- or multi-specific antibodies), may also be employed in the invention.

Binding partners (e.g., antibodies and antigen binding fragments thereof, or other peptides) useful in any embodiment of the present invention may be conjugated to detectable markers. Detectable labels suitable for use in the present invention include any composition detectable by spectroscopic, photochemical, biochemical, immunochemical, electrical, optical or chemical means, examples of which have been described above. Useful labels in the present invention include biotin for staining with labeled streptavidin conjugate, magnetic beads (e.g., Dynabeads.TM.), fluorescent dyes (e.g., fluorescein, texas red, rhodamine, green fluorescent protein, yellow fluorescent protein and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads.

Target Assays

The present invention particularly includes the use of any of the biomarkers that are differentially regulated in MS patients as described herein (including genes or their RNA or protein products), as targets for the development or identification of therapeutic compounds and strategies for the treatment of MS. More particularly, the present invention includes the use of any of the biomarkers of the invention as targets to identify compounds that regulate (up or down) the expression or activity of the biomarker or protein or gene represented by such biomarker. Such biomarkers include any biomarkers that are identified using the LC-MS method described in the Example, or any one or more of the biomarkers described in Tables 2A or 2B, or homologues, precursors or successors thereof. For example, regulatory compounds that regulate (e.g., upregulate or downregulate) the expression and/or biological activity of a target gene or its expression product (whether the product is intracellular, membrane or secreted) can be identified and/or designed using the biomarkers described herein. Alternatively, through the identification of particular genes and proteins encoded thereby that are highly regulated in patients with MS, one can use such genes and their products to further investigate the molecular or biochemical mechanisms associated with the development and progression of MS, and then design or establish assays to identify regulatory compounds that affect the molecular or biochemical mechanism with the goal of providing a therapeutic benefit to the patient.

For example, one embodiment of the present invention relates to methods for identifying compounds that regulate the expression or activity of at least one of the biomarkers described herein. Preferably, such compounds can be used to further study mechanisms associated with MS or more preferably, serve as a therapeutic agent for use in the treatment or prevention of at least one symptom or aspect of MS, or as a lead compound for the development of such a therapeutic agent. Once a biomarker has been identified as a target according to the present invention, an assay can be used for screening and selecting a chemical compound or a biological compound having regulatory activity as a candidate reagent or therapeutic based on the ability of the compound to regulate the expression or activity of the target biomarker. Reference herein to regulating a target, can refer to one or both of regulating transcription of a target gene and regulating the translation and/or activity of its corresponding expression product. Such a compound can be referred to herein as therapeutic compound, in one embodiment. For example, a cell line that naturally expresses the gene of interest or has been transfected with the gene (or suitable portions or derivatives thereof for assaying putative regulatory compounds) or other recombinant nucleic acid molecule encoding the protein of interest is incubated with various compounds, also referred to as candidate compounds, test compounds, or putative regulatory compounds. Regulation of the expression of the gene of interest or regulation of the activities of its encoded product (e.g., biological activity) may be used to identify a regulatory compound. Regulatory compounds identified in this manner can then be re-tested, if desired, in other assays (e.g., for usefulness as therapeutic compounds) to confirm their activities with regard to the target biomarker or a cellular or other activity related thereto.

In one aspect of this method of the invention, the identification of compounds that increase the expression or activity of those biomarkers identified herein that are down-regulated in the serum or CSF of patients with MS as compared to the serum or CSF of non-MS controls, are predicted to be useful as therapeutic reagents or lead compounds therefore in the prevention and treatment of MS. Similarly, the identification of compounds that decrease the expression or activity of those biomarkers identified herein that are upregulated in serum or CSF of patients with MS as compared to serum or CSF of non-MS controls, are predicted to be useful as regulatory reagents or lead compounds therefore that may have benefit in the prevention and treatment of MS.

For example one embodiment of the present invention relates to a method of using the differentially expressed biomarkers described herein as a target to identify a regulatory compound for regulation of a biological function associated with the protein represented by that biomarker or the gene encoding such protein. In one embodiment, such a method includes: (a) contacting a test compound with a biomarker of the invention; and (b) identifying compounds that regulate (upregulate or downregulate) the expression or activity of the biomarker, wherein compounds that regulate the expression or activity of the biomarker as compared to in the absence of the compound are identified as a putative therapeutic compound or lead compound with the potential to treat multiple sclerosis. In another embodiment, such a method includes: (a) contacting a test compound with a biomarker of the invention; and (b) identifying compounds that: (i) increase the expression or activity of the biomarker if the expression of the biomarker is downregulated in the serum or cerebrospinal fluid of patients with MS as compared to the expression or activity of the biomarker in the serum or cerebrospinal fluid of non-MS controls; or (ii) decrease the expression or activity of the biomarker if the expression of the biomarker is upregulated in the serum or cerebrospinal fluid of patients with MS as compared to the expression or activity of the biomarker in the serum or cerebrospinal fluid of non-MS controls. In another aspect, such a method can include the steps of: (a) contacting a test compound with a cell that expresses the target biomarker or a useful or biologically active portion, fragment or homologue thereof (i.e., useful being any portion of a gene, transcript or protein that can be used to identify a compound as discussed herein); and (b) identifying compounds that regulate the expression or activity of the gene or protein. In yet another embodiment, the method includes the steps of: (a) contacting a test compound with a biomarker of the invention; and (b) identifying compounds that bind to the biomarker. In this aspect, test compound that bind to the biomarker can be further evaluated to identify those compounds that also regulate the expression and/or biological activity of the biomarker.

In general, the biological activity or biological action of a protein refers to any function(s) exhibited or performed by the protein that is ascribed to the naturally occurring form of the protein as measured or observed in vivo (i.e., in the natural physiological environment of the protein) or in vitro (i.e., under laboratory conditions). Modifications, activities or interactions which result in a decrease in protein expression or a decrease in the activity of the protein, can be referred to as inactivation (complete or partial), down-regulation, reduced action, or decreased action or activity of a protein. Similarly, modifications, activities or interactions which result in an increase in protein expression or an increase in the activity of the protein, can be referred to as amplification, overproduction, activation, enhancement, up-regulation or increased action of a protein. The biological activity of a protein according to the invention can be measured or evaluated using any assay for the biological activity of the protein as known in the art. Such assays can include, but are not limited to, binding assays, assays to determine internalization of the protein and/or associated proteins, enzyme assays, cell signal transduction assays (e.g., phosphorylation assays), and/or assays for determining downstream cellular events that result from activation or binding of the cell surface protein (e.g., expression of downstream genes, production of various biological mediators, etc.).

According to the present invention, a biologically active fragment or homologue of a gene, nucleic acid transcript or derivative thereof, or protein maintains the ability to be useful in a method of the present invention. Therefore, the biologically active fragment or homologue maintains the ability to be used to identify regulators (e.g., inhibitors) of the native gene or protein when, for example, the biologically active fragment or homologue is expressed by a cell or used in another assay format. Therefore, the biologically active fragment or homologue has a structure that is sufficiently similar to the structure of the native gene or protein that a regulatory compound can be identified by its ability to bind to and/or regulate the expression or activity of the fragment or homologue in a manner consistent with the regulation of the native gene or protein.

As used herein, the term “expression”, when used in connection with detecting the expression of a biomarker of the present invention, can refer to detecting transcription of the gene encoding a biomarker protein and/or to detecting translation of the biomarker protein. To detect expression of a biomarker refers to the act of actively determining whether a biomarker is expressed or not. This can include determining whether the biomarker expression is upregulated as compared to a control, downregulated as compared to a control, or substantially unchanged as compared to a control. Therefore, the step of detecting expression does not require that expression of the biomarker actually is upregulated or downregulated, but rather, can also include detecting no expression of the biomarker or detecting that the expression of the biomarker has not changed or is not different (i.e., detecting no significant expression of the biomarker or no significant change in expression of the biomarker as compared to a control).

Compounds to be screened in the methods of the invention include known organic compounds such as peptides (e.g., products of peptide libraries), oligonucleotides, carbohydrates, synthetic organic molecules (e.g., products of chemical combinatorial libraries), and antibodies. Compounds may also be identified using rational drug design relying on the structure of the product of a gene or polynucleotide. Such methods are known to those of skill in the art and involve the use of three-dimensional imaging software programs. For example, various methods of drug design, useful to design or select mimetics or other therapeutic compounds useful in the present invention are disclosed in Maulik et al., 1997, Molecular Biotechnology: Therapeutic Applications and Strategies, Wiley-Liss, Inc., which is incorporated herein by reference in its entirety.

As used herein, a mimetic refers to any peptide or non-peptide compound that is able to mimic the biological action of a naturally occurring peptide, often because the mimetic has a basic structure that mimics the basic structure of the naturally occurring peptide and/or has the salient biological properties of the naturally occurring peptide. Mimetics can include, but are not limited to: peptides that have substantial modifications from the prototype such as no side chain similarity with the naturally occurring peptide (such modifications, for example, may decrease its susceptibility to degradation); anti-idiotypic and/or catalytic antibodies, or fragments thereof; non-proteinaceous portions of an isolated protein (e.g., carbohydrate structures); or synthetic or natural organic molecules, including nucleic acids and drugs identified through combinatorial chemistry, for example. Such mimetics can be designed, selected and/or otherwise identified using a variety of methods known in the art.

A mimetic can be obtained, for example, from molecular diversity strategies (a combination of related strategies allowing the rapid construction of large, chemically diverse molecule libraries), libraries of natural or synthetic compounds, in particular from chemical or combinatorial libraries (i.e., libraries of compounds that differ in sequence or size but that have the similar building blocks) or by rational, directed or random drug design. See for example, Maulik et al., supra.

In a molecular diversity strategy, large compound libraries are synthesized, for example, from peptides, oligonucleotides, carbohydrates and/or synthetic organic molecules, using biological, enzymatic and/or chemical approaches. The critical parameters in developing a molecular diversity strategy include subunit diversity, molecular size, and library diversity. The general goal of screening such libraries is to utilize sequential application of combinatorial selection to obtain high-affinity ligands for a desired target, and then to optimize the lead molecules by either random or directed design strategies. Methods of molecular diversity are described in detail in Maulik, et al., ibid.

Maulik et al. also disclose, for example, methods of directed design, in which the user directs the process of creating novel molecules from a fragment library of appropriately selected fragments; random design, in which the user uses a genetic or other algorithm to randomly mutate fragments and their combinations while simultaneously applying a selection criterion to evaluate the fitness of candidate ligands; and a grid-based approach in which the user calculates the interaction energy between three dimensional receptor structures and small fragment probes, followed by linking together of favorable probe sites.

Designing a compound for testing in a method of the present invention can include creating a new chemical compound or searching databases of libraries of known compounds (e.g., a compound listed in a computational screening database containing three dimensional structures of known compounds). Designing can also be performed by simulating chemical compounds having substitute moieties at certain structural features. The step of designing can include selecting a chemical compound based on a known function of the compound. A preferred step of designing comprises computational screening of one or more databases of compounds in which the three dimensional structure of the compound is known and is interacted (e.g., docked, aligned, matched, interfaced) with the three dimensional structure of a target by computer (e.g. as described by Humblet and Dunbar, Animal Reports in Medicinal Chemistry, vol. 28, pp. 275-283, 1993, M Venuti, ed., Academic Press). Methods to synthesize suitable chemical compounds are known to those of skill in the art and depend upon the structure of the chemical being synthesized. Methods to evaluate the bioactivity of the synthesized compound depend upon the bioactivity of the compound (e.g., inhibitory or stimulatory).

Candidate compounds identified or designed by the methods of the invention can be synthesized using techniques known in the art, and depending on the type of compound. Synthesis techniques for the production of non-protein compounds, including organic and inorganic compounds are well known in the art. For example, for smaller peptides, chemical synthesis methods are preferred. For example, such methods include well known chemical procedures, such as solution or solid-phase peptide synthesis, or semi-synthesis in solution beginning with protein fragments coupled through conventional solution methods. Such methods are well known in the art and may be found in general texts and articles in the area such as: Merrifield, 1997, Methods Enzymol. 289:3-13; Wade et al., 1993, Australas Biotechnol. 3(6):332-336; Wong et al., 1991, Experientia 47(11-12):1123-1129; Carey et al., 1991, Ciba Found Symp. 158:187-203; Plaue et al., 1990, Biologicals 18(3):147-157; Bodanszky, 1985, Int. J. Pept. Protein Res. 25(5):449-474; or H. Dugas and C. Penney, BIOORGANIC CHEMISTRY, (1981) at pages 54-92, all of which are incorporated herein by reference in their entirety. For example, peptides may be synthesized by solid-phase methodology utilizing a commercially available peptide synthesizer and synthesis cycles supplied by the manufacturer. One skilled in the art recognizes that the solid phase synthesis could also be accomplished using the FMOC strategy and a TFA/scavenger cleavage mixture. A compound that is a protein or peptide can also be produced using recombinant DNA technology and methods standard in the art, particularly if larger quantities of a protein are desired.

As used herein, the term “test compound”, “putative inhibitory compound” or “putative regulatory compound” refers to compounds having an unknown or previously unappreciated regulatory activity in a particular process. As such, the term “identify” with regard to methods to identify compounds is intended to include all compounds, the usefulness of which as a regulatory compound for the purposes of regulating the expression or activity of a target biomarker or otherwise regulating some activity that may be useful in the study or treatment of MS is determined by a method of the present invention.

In one embodiment of the invention, regulatory compounds are identified by exposing a target gene to a test compound; measuring the expression of a target; and selecting a compound that regulates (up or down) the expression of the target. For example, the putative regulatory compound can be exposed to a cell that expresses the target gene (endogenously or recombinantly). A preferred cell to use in an assay includes a mammalian cell that either naturally expresses the target gene or has been transformed with a recombinant form of the target gene, such as a recombinant nucleic acid molecule comprising a nucleic acid sequence encoding the target protein or a useful fragment thereof. Methods to determine expression levels of a gene are well known in the art and are described below.

The conditions under which a cell, cell lysate, nucleic acid molecule or protein of the present invention is exposed to or contacted with a putative regulatory compound, such as by mixing, are any suitable culture or assay conditions. In the case of a cell-based assay, the conditions include an effective medium in which the cell can be cultured or in which the cell lysate can be evaluated in the presence and absence of a putative regulatory compound. Cells of the present invention can be cultured in a variety of containers including, but not limited to, tissue culture flasks, test tubes, microtiter dishes, and petri plates. Culturing is carried out at a temperature, pH and carbon dioxide content appropriate for the cell. Such culturing conditions are also within the skill in the art. Cells are contacted with a putative regulatory compound under conditions which take into account the number of cells per container contacted, the concentration of putative regulatory compound(s) administered to a cell, the incubation time of the putative regulatory compound with the cell, and the concentration of compound administered to a cell. Determination of effective protocols can be accomplished by those skilled in the art based on variables such as the size of the container, the volume of liquid in the container, conditions known to be suitable for the culture of the particular cell type used in the assay, and the chemical composition of the putative regulatory compound (i.e., size, charge etc.) being tested. A preferred amount of putative regulatory compound(s) can comprise between about 1 nM to about 10 mM of putative regulatory compound(s) per well of a 96-well plate.

To detect expression of a target refers to the act of actively determining whether a target is expressed or not. This can include determining whether the target expression is upregulated as compared to a control, downregulated as compared to a control, or unchanged as compared to a control. Therefore, the step of detecting expression does not require that expression of the target actually is upregulated or downregulated, but rather, can also include detecting that the expression of the target has not changed (i.e., detecting no expression of the target or no change in expression of the target).

Expression of genes/transcripts and/or proteins encoded by the genes represented by biomarkers of the invention is measured by any of a variety of known methods in the art. In general, expression of a nucleic acid molecule (e.g., DNA or RNA) can be detected by any suitable method or technique of measuring or detecting gene or polynucleotide sequence or expression. Such methods include, but are not limited to, polymerase chain reaction (PCR), reverse transcriptase-PCR (RT-PCR), in situ PCR, quantitative PCR (q-PCR), in situ hybridization, Southern blot, Northern blot, sequence analysis, microarray analysis, detection of a reporter gene, or other DNA/RNA hybridization platforms. For RNA expression, preferred methods include, but are not limited to: extraction of cellular mRNA and Northern blotting using labeled probes that hybridize to transcripts encoding all or part of one or more of the genes of this invention; amplification of mRNA expressed from one or more of the genes represented by biomarkers of this invention using gene-specific primers, polymerase chain reaction (PCR), quantitative PCR (q-PCR), and reverse transcriptase-polymerase chain reaction (RT-PCR), followed by quantitative detection of the product by any of a variety of means; extraction of total RNA from the cells, which is then labeled and used to probe cDNAs or oligonucleotides encoding all or part of the genes of this invention, arrayed on any of a variety of surfaces; in situ hybridization; and detection of a reporter gene. The term “quantifying” or “quantitating” when used in the context of quantifying transcription levels of a gene can refer to absolute or to relative quantification. Absolute quantification may be accomplished by inclusion of known concentration(s) of one or more target nucleic acids and referencing the hybridization intensity of unknowns with the known target nucleic acids (e.g. through generation of a standard curve). Alternatively, relative quantification can be accomplished by comparison of hybridization signals between two or more genes, or between two or more treatments to quantify the changes in hybridization intensity and, by implication, transcription level.

Methods to measure protein expression levels of selected biomarkers of this invention, include, but are not limited to: Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, liquid chromatography mass spectrometry (LC-MS), matrix-assisted laser desorption/ionization time-of-flight (MALDI-TOF) mass spectrometry, microcytometry, microarray, microscopy, fluorescence activated cell sorting (FACS), flow cytometry, and assays based on a property of the protein including but not limited to DNA binding, ligand binding, or interaction with other protein partners.

In another embodiment of the invention, putative regulatory compounds are identified by exposing a target to a candidate compound; measuring the binding of the candidate compound to the target; and selecting a compound that binds to the target at a desired concentration, affinity, or avidity. In a preferred embodiment, the assay is performed under conditions conducive to promoting the interaction or binding of the compound to the target. One of skill in the art can determine such conditions based on the target and the compound being used in the assay. In one embodiment, a BIAcore machine can be used to determine the binding constant of a complex between the target protein (a protein encoded by the target gene) and a natural ligand in the presence and absence of the candidate compound. For example, the target protein or a ligand binding fragment thereof can be immobilized on a substrate. A natural or synthetic ligand is contacted with the substrate to form a complex. The dissociation constant for the complex can be determined by monitoring changes in the refractive index with respect to time as buffer is passed over the chip (O'Shannessy et al. Anal. Biochem. 212:457-468 (1993); Schuster et al., Nature 365:343-347 (1993)). Contacting a candidate compound at various concentrations with the complex and monitoring the response function (e.g., the change in the refractive index with respect to time) allows the complex dissociation constant to be determined in the presence of the test compound and indicates whether the candidate compound is either an inhibitor or an agonist of the complex. Alternatively, the candidate compound can be contacted with the immobilized target protein at the same time as the ligand to see if the candidate compound inhibits or stabilizes the binding of the ligand to the target protein.

Other suitable assays for measuring the binding of a candidate compound to a target protein or for measuring the ability of a candidate compound to affect the binding of the target protein to another protein or molecule include, but are not limited to, Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microarray, microscopy, fluorescence activated cell sorting (FACS), and flow cytometry. Other assays include those that are suitable for monitoring the effects of protein binding, including, but not limited to, cell-based assays such as: cytokine secretion assays, or intracellular signal transduction assays that determine, for example, protein or lipid phosphorylation, mediator release or intracellular Ca⁺⁺ mobilization.

In yet another embodiment, putative regulatory compounds are identified by exposing a target protein of the present invention (or a cell expressing the protein naturally or recombinantly) to a candidate compound and measuring the ability of the compound to inhibit or enhance a biological activity of the protein. In one embodiment, the biological activity of a protein encoded by the target gene is measured by measuring the amount of product generated in a biochemical reaction mediated by the protein encoded by the target gene. In still another embodiment, the activity of the protein encoded by the target gene is measured by measuring the amount of substrate generated in a biochemical reaction mediated by the protein encoded by the target gene. In another embodiment, a biological activity is measured by measuring a specific event in a cell-based assay, such as release or secretion of a biological mediator or compound that is regulated by the activity of the target protein, measuring intracellular signal transduction assays that determine, for example, protein or lipid phosphorylation, mediator release or intracellular Ca⁺⁺ mobilization. Preferably, the activity of the protein is measured in the presence and absence of the candidate compound, or in the presence of another suitable control compound.

In one embodiment of the invention, when the protein encoded by a target gene is an enzyme, a therapeutic compound is identified by exposing the enzyme encoded by a target gene to a test compound; measuring the activity of the enzyme encoded by the target gene in the presence and absence of the compound; and selecting a compound that down-regulates or inhibits the activity of the enzyme encoded by the target gene. Methods to measure enzymatic activity are well known to those skilled in the art and are selected based on the identity of the enzyme being tested. For example, if the enzyme is a kinase, phosphorylation assays can be used.

Preferably, methods used to identify therapeutic compounds are customized for each target gene or product. For example, if the target product is an enzyme, then the enzyme will be expressed in cell culture and purified. The enzyme will then be screened in vitro against therapeutic compounds to look for inhibition of that enzymatic activity. If the target is a non-catalytic protein, then it will also be expressed and purified. Therapeutic compounds will then be tested for their ability to regulate, for example, the binding of a site-specific antibody or a target-specific ligand to the target product.

In a preferred embodiment, therapeutic compounds that bind to target products are identified, then those compounds can be further tested in biological assays that test for other desirable characteristics and activities, such as utility as a reagent for the study of MS or utility as a therapeutic compound for the prevention or treatment of MS.

If a suitable therapeutic compound is identified using the methods and genes of the present invention, a composition can be formulated. A composition, and particularly a therapeutic composition, of the present invention generally includes the therapeutic compound and a carrier, and preferably, a pharmaceutically acceptable carrier.

Diagnostic Methods

Accordingly, in one embodiment of the present invention, the genes identified as being regulated in the serum or CSF of patients with MS can be used as endpoints or biomarkers in a diagnostic or prognostic assay for MS. This method generally includes the steps of: (a) detecting in a biological sample from a patient to be tested (e.g., a patient suspected of having MS) the level of expression of at least one biomarker chosen from a panel of biomarkers whose expression in this type of biological sample has been associated with MS as measured by either upregulation or downregulation of biomarker expression in the biological sample from patients with MS, as compared to the level of expression of the biomarkers in the same type of biological sample from non-MS control individuals; (b) comparing the level of expression of the biomarker or biomarkers detected in the patient sample to a level of expression of the biomarker or biomarkers that has been associated with MS and a level of expression of the biomarker or biomarkers that has been associated with non-MS controls; and (c) diagnosing MS in the patient if the expression level of the biomarker or biomarkers in the patient sample is statistically more similar to the expression level of the biomarker or biomarkers that has been associated with MS than the expression level of the biomarker or biomarkers that has been associated with the non-MS controls. It is to be understood that the present invention expressly covers additional biomarkers that can be identified using substantially the same techniques used to identify the biomarkers in Tables 2A and 2B and that any of such additional biomarkers can be used in the methods and products described herein for the biomarkers in Tables 2A or 2B. Any reference to database Accession numbers or other information regarding the biomarkers in Tables 2A or 2B is hereby incorporated by reference in its entirety.

In addition, the present invention will also be useful for the validation in other studies of the clinical significance of many of the specific biomarkers described herein, as well as the identification of preferred biomarker profiles, highly sensitive biomarkers, and targets for the design of novel therapeutic products and strategies.

Diagnostic assays include assays that determine whether a patient has overt MS or preclinical stage MS, and can include a more specific diagnosis of a particular form of MS (i.e., relapsing/remitting MS, secondary progressive MS, progressive/relapsing MS, or primary progressive MS (PPMS)). Prognostic assays can be used to stage a patient's development of MS, predict a patient's outcome or disease progression, and/or monitor the effectiveness of various treatment protocols on MS.

According to the present invention, the method includes the step of detecting the expression of at least one, and preferably more than one (e.g., 2, 3, 4, 5, 6, . . . and so on, in increments of whole numbers up to all of the biomarkers) of the biomarkers that have now been shown to be differentially regulated in serum or CSF of patients with MS by the present inventor. As discussed above, the step of detecting expression does not require that expression of the gene actually is upregulated or downregulated, but rather, can also include detecting no expression of the gene or detecting that the expression of the gene has not changed or is not different (i.e., detecting no significant expression of the gene or no significant change in expression of the gene as compared to a control).

In a preferred embodiment, the step of detecting includes detecting the expression of at least 2 biomarkers, and preferably at least 3 biomarkers, and more preferably at least 4 biomarkers, and more preferably at least 5 biomarkers, and more preferably at least 6 biomarkers, and more preferably at least 7 biomarkers, and more preferably at least 8 biomarkers, and more preferably at least 9 biomarkers, and more preferably at least 10 biomarkers, and more preferably at least 11 biomarkers, and more preferably at least 12 biomarkers, and more preferably at least 13 biomarkers, and more preferably at least 14 biomarkers, and more preferably at least 15 biomarkers, and more preferably at least 20 biomarkers, and more preferably at least 25 biomarkers, and more preferably at least 50 biomarkers, and more preferably at least 75 biomarkers, and more preferably at least 100 biomarkers, and so on, in whole integer increments (i.e., 1, 2, 3 . . . . 10, 11, 12, . . . . 35, 36, 37, . . . 56, 57, 58, . . . 98, 99, 100, . . . ), up to detecting expression of all of the biomarkers that are identified as differentially expressed in MS patients and as described herein. Analysis of a number of biomarkers greater than one can be accomplished simultaneously, sequentially, or cumulatively. As discussed above, it is preferred that several to most of the biomarkers be detected in the present methods, as the accuracy of the method improves as the number of biomarkers detected increases. However, it is to be understood that in some circumstances, it may be desirable and sufficient to detect the expression of only one or a few biomarkers.

In the diagnostic or prognostic method of the present invention, the biomarker(s) to be detected are preferably selected from the biomarkers described in one or both of Tables 2A and 2B, and include any combination of biomarkers from these Tables. These tables and biomarkers have been discussed above in detail and disclose biomarkers that the present inventor has discovered to be selectively regulated in the CSF of patients with MS. More specifically, these tables disclose the manner in which the biomarkers are regulated (e.g., upregulated or downregulated) in a patient with MS as compared to a non-MS control. It is not mandatory that a given assay be restricted to the detection of all of the various biomarkers in a single table, or to at least one biomarker in each table. In addition, one may choose also to detect other biomarkers that are believed to be useful in the evaluation of a patient for MS, and therefore, the present method is not limited exclusively to detection of the biomarkers identified herein or for which a method for identification is described herein, although the invention is primarily directed to the detection of one or more of these biomarkers and includes the detection of at least one or more of these biomarkers. In addition, provided with this disclosure, one of skill in the art may proceed to identify additional biomarkers that are differentially regulated in the CSF or serum of patients with MS, and detection of any of such biomarkers may be used in the methods of the present invention, including in combination with detection of any of the specific biomarkers disclosed herein.

In one aspect, it may be desirable to preferentially select those biomarkers for detection that are particularly highly regulated in patients with MS in that they display the largest increases or decreases in expression levels in patients as compared to non-MS controls. The detection of such biomarkers can be advantageous because the endpoint may be more clear and require less quantitation.

According to the present invention, a “baseline” or “control” can include a normal or negative control and/or a disease or positive control, against which a test level of biomarker expression can be compared. Therefore, it can be determined, based on the control or baseline level of biomarker expression, whether a sample to be evaluated for MS has a measurable difference or substantially no difference in biomarker expression, as compared to the baseline level. In one aspect, the baseline control is a indicative of the level of biomarker expression as expected in the CSF or serum of a normal (e.g., healthy, negative control, non-MS) patient. Therefore, the term “negative control” used in reference to a baseline level of biomarker expression typically refers to a baseline level of expression from a population of individuals which is believed to be normal (i.e., not having or developing MS). In some embodiments of the invention, it may also be useful to compare the biomarker expression in a test sample to a baseline that has previously been established from a patient or population of patients with MS. Such a baseline level, also referred to herein as a “positive control”, refers to a level of biomarker expression established in samples from one or preferably a population of individuals who had been positively diagnosed with MS.

In one embodiment, when the goal is to monitor the progression or regression of MS in a patient, for example, to monitor the efficacy of treatment of the disease or to determine whether a patient that appears to be predisposed to the disease begins to develop the disease, one baseline control can include the measurements of biomarker expression in a sample from the patient that was taken from a prior test in the same patient. In this embodiment, a new sample is evaluated periodically (e.g., at annual or more regular physicals), and any changes in biomarker expression in the patient sample as compared to the prior measurement and most typically, also with reference to the above-described normal and/or positive controls, are monitored. Monitoring of a patient's biomarker expression profile can be used by the clinician to prescribe or modify treatment for the patient based on whether any differences in biomarker expression in the sample is indicated.

In a preferred embodiment, the control or baseline levels of biomarker expression are obtained from samples collected from “matched individuals”. According to the present invention, the phrase “matched individuals” refers to a matching of the control individuals on the basis of one or more characteristics, such as gender, age, race, or any relevant biological or sociological factor that may affect the baseline of the control individuals and the patient (e.g., preexisting conditions, consumption of particular substances, levels of other biological or physiological factors). The number of matched individuals from whom control samples must be obtained to establish a suitable control level (e.g., a population) can be determined by those of skill in the art, but should be statistically appropriate to establish a suitable baseline for comparison with the patient to be evaluated (i.e., the test patient). The values obtained from the control samples are statistically processed using any suitable method of statistical analysis to establish a suitable baseline level using methods standard in the art for establishing such values. It will be appreciated by those of skill in the art that a baseline need not be established for each assay as the assay is performed but rather, a baseline can be established by referring to a form of stored information regarding a previously determined control level of biomarker expression. Such a form of stored information can include, for example, but is not limited to, a reference chart, listing or electronic file of population or individual data regarding “normal” (negative control) or MS-positive biomarker expression; a medical chart for the patient recording data from previous evaluations; or any other source of data regarding control biomarker expression that is useful for the patient to be diagnosed or evaluated. Methods for detection of the expression of both polynucleotides (e.g., genes) and polypeptides encoded thereby have been described above.

In general, typical biological samples useful in the present method include, but are not limited to, any cell sample, tissue sample or body fluid sample, the body fluid sample including, but not limited to, cerebrospinal fluid (CSF), serum, plasma, blood, urine, prostatic fluid, saliva or fluid from any suitable tissue. In a preferred embodiment, the biological sample is a CSF sample.

The method of the present invention includes a step of comparing the results of detecting the expression of the one or more biomarkers that are selectively regulated in patients with MS as compared to a control (baseline, non-MS control or patient with an alternate form of MS) in order to determine whether there is any observed change or difference in expression of each biomarker in the patient as compared to the control. One can determine whether a sample from a test patient has a biomarker expression profile that is statistically substantially similar to the profile of biomarker expression of a patient with MS, or in one embodiment, with a particular form of MS, or whether a profile of biomarker expression in the sample of the test patient is statistically more similar to the negative or normal, non-disease control.

According to the present invention, an expression profile is substantially similar to a given profile of expression established for a group (e.g., MS group, non-MS control group) if the expression profile of the biomarker or biomarkers detected (including the identity of the biomarker, the manner in which expression is regulated, and/or the level of expression of the biomarker) is similar enough to the expected result so as to be statistically significant (i.e., with at least a 95% confidence level, or p<0.05, and more preferably, with a confidence level of p<0.01, and even more preferably, with a confidence level of p<0.005, and even more preferably, with a confidence level of p<0.001). Software programs are available in the art that are capable of analyzing the expression of multiple biomarkers and determining whether differences from a control are significant or not significant. Statistical analysis methods are well-known known in the art.

By way of example, detection of the regulation of the expression of a biomarker in the “manner” associated with the established group, at a minimum, refers to the detection of the regulation of a biomarker that has now been shown by the present inventor to be selectively regulated in CSF or serum of patients having MS, in the same direction (i.e., upregulation or downregulation) and at a similar or comparable level, as compared to a normal or baseline control established for the expression of that biomarker. Preferably, a biomarker identified as being upregulated or downregulated, as compared to a baseline control, is regulated in the same direction as the level of expression of the biomarker that is seen in established or confirmed patients with MS as compared to a normal control. In other words, if “biomarker X” is upregulated in patients with MS as compared to a normal control based on the inventor's discovery presented herein, then one determines whether the expression of biomarker X is upregulated in a patient test sample as compared to a normal control, or whether the expression of biomarker X is more similar to the level of expression of the normal control. In one aspect of the invention, a biomarker is identified as being upregulated or downregulated as compared to a baseline control according to the invention is regulated in the same direction and to at least about 10%, and more preferably at least 20%, and more preferably at least 25%, and more preferably at least 30%, and more preferably at least 35%, and more preferably at least 40%, and more preferably at least 45%, and more preferably at least 50%, and preferably at least 55%, and more preferably at least 60%, and more preferably at least 65%, and more preferably at least 70%, and more preferably at least 75%, and more preferably at least 80%, and more preferably at least 85%, and more preferably at least 90%, and more preferably at least 95%, or even higher (e.g., above 100%) of the level of expression of the biomarker that is seen in established or confirmed patients with MS. Statistical significance should be at least p<0.05, and more preferably, at least p<0.01, and more preferably, p<0.005, and even more preferably, p<0.001. As discussed above, one of skill in the art can use software programs available in the art which use algorithms to analyze biomarker expression profiles and identify significant differences among samples and controls. In addition, one of skill in the art can apply various types of statistical analyses to validate the results of the methods described herein.

It will be appreciated by those of skill in the art that differences between the expression of biomarkers in the serum or CSF of patients with MS and without MS may be small or large. Some small differences may be very reproducible and therefore are preferred for use in the diagnostic and prognostic methods of the invention. For other purposes, large differences may be desirable for ease of detection of the regulatory activity. It will therefore be appreciated that the exact boundary between a positive diagnosis and a negative diagnosis can shift, depending on the goal of the screening assay, the patient samples, the number of biomarkers to be screened and the baseline controls used. For some assays, a given patient may be sampled over time to detect the efficacy of a treatment, and so changes in biomarker expression from a disease state toward a normal state may be detected. In this case, the patient may still be positive for a given form of MS as compared to a normal, disease-free control, but may show a shift toward the normal control biomarker expression profile if treatment is successful. In addition, the technique being used for detection as well as on the number of biomarkers which are being tested may impact how the assay is evaluated by those of skill in the art.

The profile of genes provided as a result of the screening of biological samples of a patient can be used by the patient or physician for decision-making regarding the usefulness of therapies for MS in general. The profile can be used to estimate how the disease is likely to respond and progress in any individual patient. Clinical trials can be developed to correlate the relationship between MS-regulated genes and the biological behavior of the diseased tissues, including in response to particular treatments for MS.

Assay Kits

The present invention also provides assay kits that are suitable for the performance of any method described herein and/or the detection of any of the biomarkers for MS that are described herein. The assay kit preferably contains at least one reagent that is suitable for detecting the expression or activity of a biomarker of the present invention in a test sample (e.g., serum or CSF), and preferably includes a probe, PCR primers, an antibody or antigen binding fragment thereof, peptides, binding partners, aptamers, enzymes, enzyme substrates and small molecules that bind to or otherwise identify a biomarker of the invention. The kit can include any reagent needed to perform a diagnostic method envisioned herein or to perform a target-based assay envisioned herein. In one embodiment, the kit can contain a means for detecting a control marker characteristic of a cell type in the test sample. The kit can also include suitable reagents for the detection of and/or for the labeling of positive or negative controls, wash solutions, dilution buffers and the like. The kit can also include a set of written instructions for using the kit and interpreting the results.

The means for detecting of the assay kit of the present invention can be conjugated to a detectable tag or detectable label. Such a tag can be any suitable tag which allows for detection of the reagents used to detect the gene or protein of interest and includes, but is not limited to, any composition or label detectable by spectroscopic, photochemical, electrical, optical or chemical means.

In addition, the means for detecting of the assay kit of the present invention can be immobilized on a substrate. Such a substrate can include any suitable substrate for immobilization of a detection reagent such as would be used in any of the previously described methods of detection. Briefly, a substrate suitable for immobilization of a means for detecting includes any solid support, such as any solid organic, biopolymer or inorganic support that can form a bond with the means for detecting without significantly affecting the activity and/or ability of the detection means to detect the desired target molecule. Exemplary organic solid supports include polymers such as polystyrene, nylon, phenol-formaldehyde resins, and acrylic copolymers (e.g., polyacrylamide).

Each reference or publication cited herein is incorporated herein by reference in its entirety.

The following examples are provided for the purpose of illustration and are not intended to limit the scope of the present invention.

### EXAMPLE

The following example demonstrates the identification of biomarkers useful in the present invention.

Although CSF proteins are 200-fold less concentrated than serum proteins, a sensitive yet robust method was developed for fractionation, differential quantification and identification of these proteins by liquid chromatography mass spectrometry (LC-MS). After removal of metabolites by ultrafiltration and removal of highly abundant proteins by affinity chromatography, the proteome was subjected to tryptic digestion. The resulting digested peptides were separated and analyzed on an HPLC system interfaced to an electrospray ionization time of flight (ESI-TOF) mass spectrometer. For direct differential quantification without isotope dilution, version 2.0 of the inventor's proprietary quantification software (MassView™ software) was developed, tested and validated.

Methods

MS Study Design

All afflicted individuals were relapsing-remitting MS patients (Lublin and Reingold, 1996, Neurology 46(4):907-11).

Cerebrospinal fluid (CSF): 18 controls and 18 MS-afflicted individuals were compared.

Serum: 18 controls and 18 MS-afflicted individuals were compared.

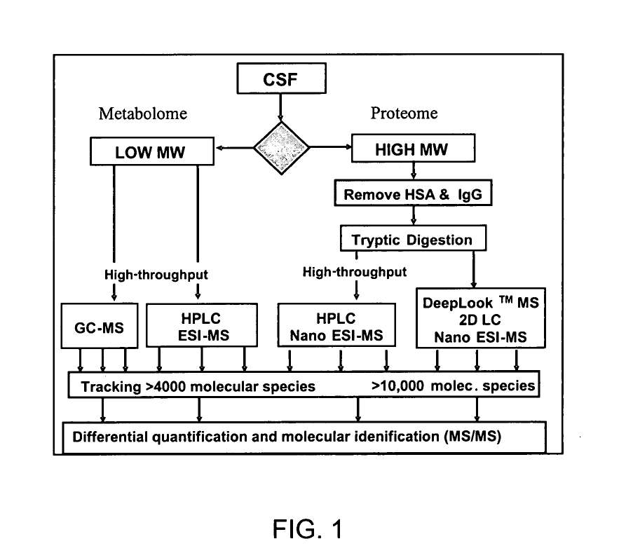

Sample Preparation and LC-MS Analysis of CSF (see FIG. 1.):

Fractionation with a 5 kD cut off spin filter
Removal of abundant proteins
Reduction and alkylation and digestion of proteins
Reversed-phase chromatography on a capillary column
Analysis on an electrospray ionization time of flight (ESI-TOF) mass spectrometer interfaced to the LC system
Only 0.5 mL of CSF required
   Quantitation of Differential Expression

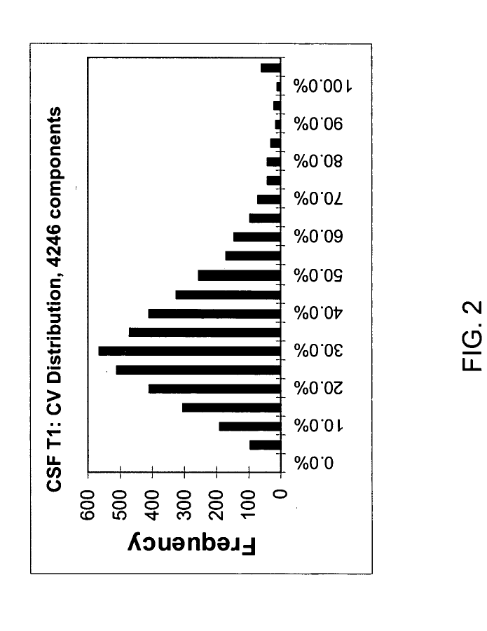

The inventor and colleagues have previously reported a method for direct quantification of molecular ions for differential profiling using proprietary quantification software (MassView™ software; Roy et al., 2004, International Journal of Mass Spectrometry; Anderle et al., 2004, Bioinformatics 20(18):3575-3582; Wang et al., 2003, Analytical Chemistry 75:4818; Hastings et al., 2002, Rapid Commumications in Mass Spectrometry 16:462.). This platform was recently improved with several new features, validated, tested and released as Version 2.0. Some salient features are:
Deisotoping of molecular ions and charge state determination using peak intensity information.
Intensity normalization across patients using the median of intensity ratios between samples.
Correction of retention time variation between chromatographic runs using dynamic time warping.
Matching of peaks between samples using their accurate masses and retention times.
Direct intensity measurement (normalized) of the molecular ions can in each sample.
T-test to find significant statistical differences between healthy controls and MS patients.
Typical coefficients of variation (CV) are 25% to 30%. FIG. 2 shows the CV distribution among 20 individuals, for 4000 ions measured directly in the CSF proteome.
   Protein Identification
The accurate masses and retention times of the molecular ions of interest were used to search for possible matches from the inventor and colleagues' proprietary protein/peptide library.
Remaining unidentified molecular ions were then subjected to targeted MS/MS experiments.

For each study, peptides from ˜200 hundred proteins were tracked, identified and quantified.

Results

For this study, proteins from 18 healthy and 18 MS-afflicted individuals were differentially quantified in both CSF and serum. The methodology and automated analysis developed by the inventor and colleagues enable the undertaking of large clinical studies in search of biomarkers. This platform was successfully applied to protein profiling in cerebrospinal fluid (CSF) from MS patients.

(1) CSF Sample Preparation: One mL of CSF was sufficient to quantify over 4000 molecular ions in each sample (not counting isotopes) with signal-to-noise ratios above 5:1. Sample processing methods were robust and reproducible.

(2) Software Development for Differential Profiling: The quantification software (MassView™ software) was further developed. New features added include improved baseline-correction, local noise elimination, isotope and charge state assignments, and component building across files. Each molecular ion (not counting isotopes) is considered a ‘component.’

(3) Differential Profiling in Multiple Sclerosis: For comparison between MS and normal samples, retention time correction and normalization were applied. For a given molecular ion, intensities in all samples (healthy and MS) were obtained by searching for that molecular ion using its accurate mass, aligned retention time and charge state in each data file. Standard statistical analyses were applied to find molecular ions that were significantly over or under expressed in MS patients.

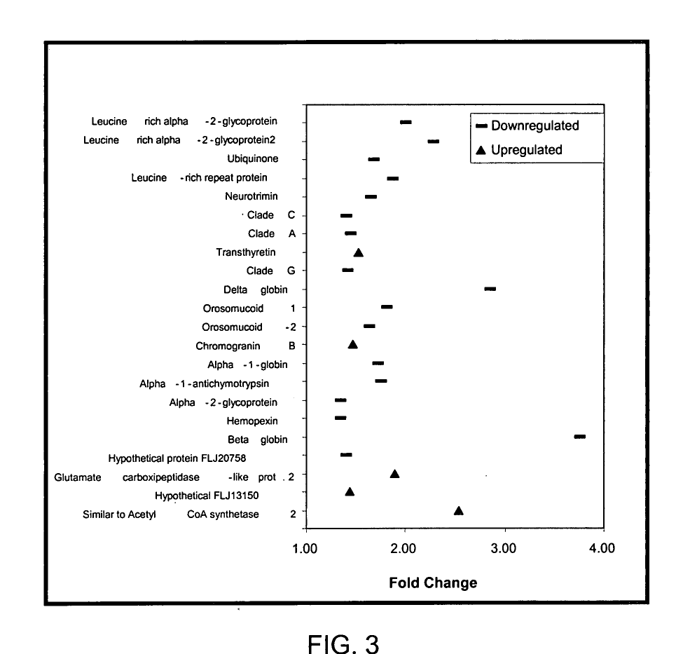

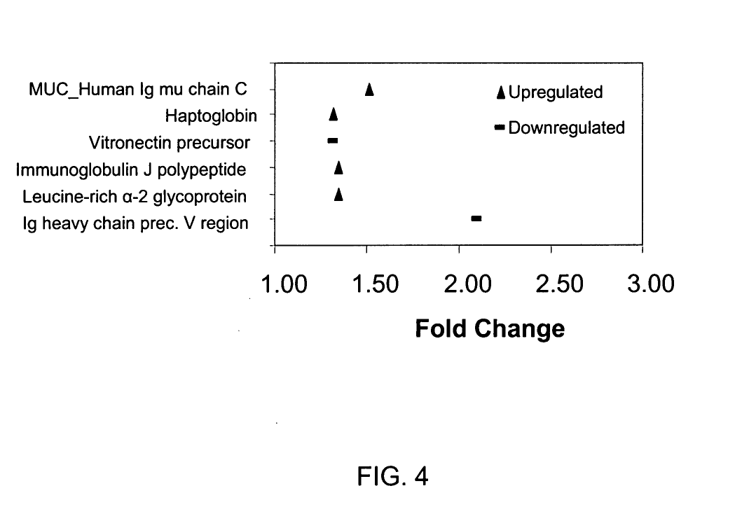

CSF proteome difference heat maps were generated (data not shown). Four hundred significant changes (p<0.05) were found between the MS and the control group. FIG. 3 shows some proteins discovered in CSF that are up- or downregulated significantly in relapsing-remitting multiple sclerosis. Using the serum samples, serum proteome difference heat maps were also created (data not shown). 313 significant changes (p<0.05) were found between MS and control groups. FIG. 4 shows some proteins discovered in serum that are up- or downregulated significantly in relapsing-remitting multiple sclerosis.

Table 1 shows a comparison of the CSF and serum proteomic profiling. The CSF profiling requires ˜100 times the volume of serum. The results showed that differential expression profiling is as robust in CSF as in serum, CVs are comparable, and in multiple sclerosis, more significant changes are observed in CSF than in serum.

TABLE 1
Comparison of CSF and Serum Proteomic Profiling.

| Fluid for differential protein profiling | CSF | | Serum | |
|---|---|---|---|---|
| Volume of fluid used for profiling | 1 | mL | 7.5 | μL |
| Total amount of protein in sample | 20 | μg | 20 | μg |

| | | |
|---|---|---|
| Total # Molecular Ions Quantified | 4162 | 7308 |
| # Significant Changes P < 0.001 | 13 | 6 |
| # Significant Changes P < 0.005 | 71 | 30 |
| # Significant Changes P < 0.01 | 120 | 63 |
| # Significant Changes P < 0.05 | 400 | 313 |
| # Absent or present in a group | 4 | 61 |
| MS Group Average % CV | 40.0% | 31.4% |
| Control Group Average % CV | 43.2% | 29.9% |
| MS Group Median % CV | 35.0% | 27.6% |
| Control Group Median % CV | 40.2% | 26.9% |

(4) Protein Identification: A unique strategy was used so that all samples did not require analysis by tandem mass spectrometry. First, a database consisting of several hundred proteins, identified in CSF by tandem mass spectrometry, was created. All molecular ions measured in this differential profiling study were matched against the library using their accurate mass and chromatographic retention times. For molecular ions that were not successfully matched, additional tandem mass spectral experiments were conducted.

The CSF proteome data obtained as discussed above was analyzed using MassView v.2.0, and the results are presented in Tables 2A (names unavailable) and 2B (names available).

TABLE 2A
CSF proteome data obtained (names unavailable)

| Comp. # | m/z | R.T.(min.) | z | M + H | Exp. Ratio | Fold Change | Trend | P-value |
|---|---|---|---|---|---|---|---|---|
| 3991 | 306.15 | 40.03 | 3 | 916.43 | 1.40 | 1.40 | Up | 5.78E−03 |
| 2298 | 322.16 | 24.38 | 2 | 643.31 | 1.30 | 1.30 | Up | 9.74E−03 |
| 100 | 327.39 | 37.48 | 4 | 1306.54 | 0.61 | −1.64 | Down | 2.05E−03 |
| 132 | 335.68 | 26.27 | 2 | 670.35 | 1.36 | 1.36 | Up | 5.84E−04 |
| 4355 | 336.86 | 28.34 | 3 | 1008.56 | 0.71 | −1.40 | Down | 3.85E−03 |
| 136 | 336.92 | 26.29 | 2 | 672.83 | 1.50 | 1.50 | Up | 3.58E−04 |
| 153 | 340.48 | 23.34 | 3 | 1019.42 | 0.51 | −1.96 | Down | 8.18E−03 |
| 4024 | 352.82 | 40.47 | 3 | 1056.44 | 1.36 | 1.36 | Up | 2.19E−04 |
| 3935 | 354.50 | 40.44 | 3 | 1061.48 | 1.31 | 1.31 | Up | 7.50E−03 |
| 240 | 358.71 | 34.93 | 2 | 716.41 | 0.47 | −2.13 | Down | 9.90E−04 |
| 261 | 361.15 | 26.40 | 3 | 1081.43 | 0.61 | −1.63 | Down | 5.01E−03 |
| 319 | 373.73 | 75.35 | 2 | 746.45 | 1.70 | 1.70 | Up | 3.34E−03 |
| 3573 | 375.71 | 49.02 | 1 | 375.71 | 1.29 | 1.29 | Up | 8.71E−03 |
| 359 | 382.54 | 36.59 | 3 | 1145.60 | 0.56 | −1.78 | Down | 4.03E−03 |
| 373 | 383.85 | 74.29 | 3 | 1149.53 | 2.07 | 2.07 | Up | 1.84E−03 |
| 376 | 384.71 | 49.04 | 4 | 1535.82 | 1.35 | 1.35 | Up | 6.69E−04 |
| 377 | 384.86 | 39.04 | 3 | 1152.56 | 0.50 | −2.00 | Down | 4.18E−04 |
| 408 | 389.17 | 74.28 | 3 | 1165.49 | 1.98 | 1.98 | Up | 9.70E−03 |
| 573 | 418.20 | 33.82 | 1 | 418.20 | 2.36 | 2.36 | Up | 4.30E−03 |
| 579 | 419.20 | 27.91 | 2 | 837.39 | 0.64 | −1.56 | Down | 9.11E−03 |
| 2989 | 421.56 | 50.62 | 3 | 1262.66 | 0.51 | −1.96 | Down | 6.04E−03 |
| 3110 | 423.73 | 46.91 | 2 | 846.45 | 1.58 | 1.58 | Up | 7.05E−03 |
| 4098 | 427.17 | 39.59 | 1 | 427.17 | 1.92 | 1.92 | Up | 9.78E−03 |
| 634 | 429.18 | 47.94 | 2 | 857.35 | 1.50 | 1.50 | Up | 3.18E−03 |
| 658 | 434.20 | 40.00 | 2 | 867.39 | 0.57 | −1.74 | Down | 4.07E−03 |
| 747 | 448.15 | 47.91 | 2 | 895.29 | 1.48 | 1.48 | Up | 5.29E−03 |
| 787 | 456.14 | 47.93 | 2 | 911.27 | 2.16 | 2.16 | Up | 7.09E−03 |
| 874 | 469.22 | 54.69 | 1 | 469.22 | 1.44 | 1.44 | Up | 1.99E−03 |
| 880 | 469.92 | 54.44 | 3 | 1407.74 | 1.33 | 1.33 | Up | 6.78E−03 |
| 982 | 482.75 | 37.78 | 2 | 964.49 | 3.73 | 3.73 | Up | 1.43E−04 |
| 4164 | 491.69 | 57.52 | 4 | 1963.74 | 2.18 | 2.18 | Up | 4.87E−03 |
| 4168 | 492.26 | 40.00 | 2 | 983.51 | 0.58 | −1.71 | Down | 3.23E−04 |
| 1041 | 493.88 | 27.11 | 3 | 1479.62 | 0.64 | −1.56 | Down | 1.45E−03 |
| 4170 | 494.76 | 33.36 | 2 | 988.51 | 0.53 | −1.90 | Down | 7.07E−03 |
| 1104 | 506.60 | 73.74 | 3 | 1517.78 | 2.56 | 2.56 | Up | 5.28E−03 |
| 4190 | 516.91 | 49.02 | 3 | 1548.71 | 1.72 | 1.72 | Up | 7.27E−03 |
| 1166 | 517.76 | 35.35 | 2 | 1034.51 | 0.57 | −1.74 | Down | 4.22E−03 |
| 1215 | 523.57 | 35.99 | 3 | 1568.69 | 2.02 | 2.02 | Up | 4.10E−03 |
| 2607 | 538.27 | 34.71 | 1 | 538.27 | 1.84 | 1.84 | Up | 9.05E−03 |
| 3237 | 540.72 | 42.62 | 4 | 2159.86 | 2.13 | 2.13 | Up | 5.80E−03 |
| 1329 | 551.60 | 62.51 | 3 | 1652.78 | 2.91 | 2.91 | Up | 1.73E−04 |
| 1333 | 552.77 | 88.79 | 2 | 1104.53 | 1.56 | 1.56 | Up | 5.45E−03 |
| 2844 | 555.28 | 80.95 | 3 | 1663.82 | 0.57 | −1.76 | Down | 4.47E−03 |
| 1453 | 573.79 | 28.09 | 2 | 1146.57 | 3.38 | 3.38 | Up | 3.18E−03 |
| 1519 | 590.32 | 58.95 | 2 | 1179.63 | 0.57 | −1.76 | Down | 4.20E−03 |
| 1529 | 593.91 | 23.71 | 3 | 1779.71 | 0.68 | −1.48 | Down | 8.96E−03 |
| 1546 | 599.40 | 54.90 | 1 | 599.40 | 2.37 | 2.37 | Up | 8.55E−03 |
| 2440 | 633.98 | 62.48 | 3 | 1899.92 | 0.38 | −2.61 | Down | 1.41E−03 |
| 1687 | 634.32 | 34.10 | 2 | 1267.63 | 1.99 | 1.99 | Up | 3.18E−03 |
| 1808 | 677.25 | 46.29 | 3 | 2029.73 | 1.43 | 1.43 | Up | 6.05E−03 |
| 4797 | 681.34 | 42.33 | 1 | 681.34 | 0.58 | −1.73 | Down | 2.72E−03 |
| 1862 | 696.92 | 44.01 | 3 | 2088.74 | 1.66 | 1.66 | Up | 4.07E−03 |
| 1895 | 704.39 | 59.35 | 1 | 704.39 | 2.89 | 2.89 | Up | 9.21E−03 |
| 2058 | 784.86 | 36.00 | 2 | 1568.71 | 2.04 | 2.04 | Up | 1.75E−03 |
| 2970 | 991.47 | 42.38 | 1 | 991.47 | 0.55 | −1.83 | Down | 4.94E−03 |
| 2259 | 1051.44 | 60.07 | 1 | 1051.44 | 2.58 | 2.58 | Up | 8.05E−03 |

TABLE 2B
CSF proteome data obtained (names available)

| Comp. # | m/z | R.T. (min.) | z | M + H | Accession # | gi # | Protein Description | Peptide | Exp. Ratio | Fold Change | Trend | P-value |
|---|---|---|---|---|---|---|---|---|---|---|---|---|
| 1123 | 510.24 | 50.88 | 3 | 1528.70 | NP_000479.1 | 4502261 | serine (or cysteine) proteinase | FATTFYQHLADSK | 0.71 | −1.41 | Down | 6.98E−03 |
| | | | | | | | inhibitor, clade C | (SEQ ID NO: 1) | | | | |
| | | | | | | | (antithrombin), member 1; | | | | | |
| | | | | | | | antithrombin III [Homo | | | | | |
| | | | | | | | sapiens] | | | | | |
| 2183 | 891.97 | 72.14 | 2 | 1782.93 | NP_001176.1 | 4502337 | alpha-2-glycoprotein 1, zinc; | EIPAWVPFDPAAQITK | 0.73 | −1.36 | Down | 6.19E−03 |
| | | | | | | | Alpha-2-glycoprotein, zinc | (SEQ ID NO: 2) | | | | |
| | | | | | | | [Homo sapiens] | | | | | |
| 788 | 456.24 | 54.43 | 3 | 1366.70 | NP_000362.1 | 4507725 | transthyretin (prealbumin, | GSPAINVAVHVFR | 1.34 | 1.34 | Up | 4.20E−04 |
| | | | | | | | amyloidosis type I); | (SEQ ID NO: 3) | | | | |
| | | | | | | | Transthyretin (prealbumin) | | | | | |
| | | | | | | | [Homo sapiens] | | | | | |
| 1600 | 613.54 | 56.63 | 4 | 2451.14 | NP_000362.1 | 4507725 | transthyretin (prealbumin, | ALGISPFHEHAEVVFTANDSGPR | 1.42 | 1.42 | Up | 1.50E−03 |
| | | | | | | | amyloidosis type I); | (SEQ ID NO: 4) | | | | |
| | | | | | | | Transthyretin (prealbumin) | | | | | |
| | | | | | | | [Homo sapiens] | | | | | |
| 2090 | 817.72 | 56.65 | 3 | 2451.14 | NP_000362.1 | 4507725 | transthyretin (prealbumin, | ALGISPFHEHAEVVFTANDSGPR | 1.48 | 1.48 | Up | 1.97E−03 |
| | | | | | | | amyloidosis type I); | (SEQ ID NO: 5) | | | | |
| | | | | | | | Transthyretin (prealbumin) | | | | | |
| | | | | | | | [Homo sapiens] | | | | | |
| 1023 | 491.02 | 56.65 | 5 | 2451.07 | NP_000362.1 | 4507725 | transthyretin (prealbumin, | ALGISPFHEHAEVVFTANDSGPR | 1.39 | 1.39 | Up | 2.98E−03 |
| | | | | | | | amyloidosis type I); | (SEQ ID NO: 6) | | | | |
| | | | | | | | Transthyretin (prealbumin) | | | | | |
| | | | | | | | [Homo sapiens] | | | | | |
| 1797 | 672.39 | 26.25 | 1 | 672.39 | NP_000362.1 | 4507725 | transthyretin (prealbumin, | VLDAVR | 1.45 | 1.45 | Up | 3.92E−03 |
| | | | | | | | amyloidosis type I); | (SEQ ID NO: 7) | | | | |
| | | | | | | | Transthyretin (prealbumin) | | | | | |
| | | | | | | | [Homo sapiens] | | | | | |
| 1825 | 683.87 | 54.43 | 2 | 1366.73 | NP_000362.1 | 4507725 | transthyretin (prealbumin, | GSPAINVAVHVFR | 1.45 | 1.45 | Up | 4.06E−03 |
| | | | | | | | amyloidosis type I); | (SEQ ID NO: 8) | | | | |
| | | | | | | | Transthyretin (prealbumin) | | | | | |
| | | | | | | | [Homo sapiens] | | | | | |
| 1664 | 628.89 | 76.16 | 5 | 3140.42 | NP_000362.1 | 4507725 | transthyretin (prealbumin, | TSESGELHGLTTEEEFVEGIYKVEIDTK | 1.33 | 1.33 | Up | 5.24E−03 |
| | | | | | | | amyloidosis type I); | (SEQ ID NO: 9) | | | | |
| | | | | | | | Transthyretin (prealbumin) | | | | | |
| | | | | | | | [Homo sapiens] | | | | | |
| 854 | 466.22 | 36.01 | 3 | 1396.64 | NP_000362.1 | 4507725 | transthyretin (prealbumin, | SPYSYSTTAVVINPKE | 3.07 | 3.07 | Up | 5.80E−03 |
| | | | | | | | amyloidosis type I); | (SEQ ID NO: 10) | | | | |
| | | | | | | | Transthyretin (prealbumin) | | | | | |
| | | | | | | | [Homo sapiens] | | | | | |
| 322 | 374.45 | 49.05 | 4 | 1494.78 | NP_000362.1 | 4507725 | transthyretin (prealbumin, | GSPAINVAVHVFRK | 1.35 | 1.35 | Up | 6.82E−03 |
| | | | | | | | amyloidosis type I); | (SEQ ID NO: 11) | | | | |
| | | | | | | | Transthyretin (prealbumin) | | | | | |
| | | | | | | | [Homo sapiens] | | | | | |
| 1065 | 498.94 | 49.04 | 3 | 1494.80 | NP_000362.1 | 4507725 | transthyretin (prealbumin, | GSPAINVAVHVFRK | 1.35 | 1.35 | Up | 8.08E−03 |
| | | | | | | | amyloidosis type I); | SEQ ID NO: 12) | | | | |
| | | | | | | | Transthyretin (prealbumin) | | | | | |
| | | | | | | | [Homo sapiens] | | | | | |
| 2258 | ###### | 76.16 | 3 | 3140.42 | NP_000362.1 | 4507725 | transthyretin (prealbumin, | TSESGELHGLTTEEEFVEGIYKVEIDTK | 1.31 | 1.31 | Up | 8.12E−03 |
| | | | | | | | amyloidosis type I); | (SEQ ID NO: 13) | | | | |
| | | | | | | | Transthyretin (prealbumin) | | | | | |
| | | | | | | | [Homo sapiens] | | | | | |
| 2036 | 773.04 | 73.18 | 3 | 2317.10 | NP_004537.1 | 4758778 | NADH dehydrogenase | SGCARTAGDGGVRHAGGGVHIEPR | 0.60 | −1.67 | Down | 4.76E−03 |
| | | | | | | | (ubiquinone) 1 beta | (SEQ ID NO: 14) | | | | |
| | | | | | | | subcomplex, 2, 8 kDa; NADH | | | | | |
| | | | | | | | dehydrogenase (ubiquinone) 1 | | | | | |
| | | | | | | | beta subcomplex, 2 (8 kD, | | | | | |
| | | | | | | | AGGG) [Homo sapiens] | | | | | |
| 1429 | 570.27 | 49.35 | 3 | 1708.79 | NP_057606.1 | 7705413 | neurotrimin [Homo sapiens] | VKVTVNYPPYISEAK | 0.60 | −1.67 | Down | 4.77E−03 |
| | | | | | | | | (SEQ ID NO: 15) | | | | |
| 1351 | 556.75 | 43.95 | 2 | 1112.49 | NP_000598.1 | 9257232 | orosomucoid 1 precursor; | SDVVYTDWK | 0.56 | −1.77 | Down | 5.65E−03 |
| | | | | | | | Orosomucoid-1 (alpha-1-acid | (SEQ ID NO: 16) | | | | |
| | | | | | | | glycoprotein-1); alpha-1-acid | | | | | |
| | | | | | | | glycoprotein 1 [Homo sapiens] | | | | | |
| 860 | 466.55 | 51.98 | 3 | 1397.63 | NP_000598.1 | 9257232 | orosomucoid 1 precursor; | HLLILRDTKTYMLAFDVNDEKNWGL | 0.60 | −1.66 | Down | 6.56E−03 |
| | | | | | | | Orosomucoid-1 (alpha-1-acid | (SEQ ID NO: 17) | | | | |
| | | | | | | | glycoprotein-1); alpha-1-acid | | | | | |
| | | | | | | | glycoprotein 1 | | | | | |
| 1478 | 580.78 | 67.97 | 2 | 1160.55 | NP_000598.1 | 9257232 | orosomucoid 1 precursor; | WFYIASAFR | 0.50 | −2.00 | Down | 7.98E−03 |
| | | | | | | | Orosomucoid-1 (alpha-1-acid | (SEQ ID NO: 18) | | | | |
| | | | | | | | glycoprotein-1); alpha-1-acid | | | | | |
| | | | | | | | glycoprotein 1 [Homo sapiens] | | | | | |
| 2397 | 495.27 | 41.20 | 2 | 989.53 | NP_443204.1 | 16418467 | leucine-rich alpha-2- | VAAGAFQGLR | 0.44 | −2.27 | Down | 2.72E−03 |
| | | | | | | | glycoprotein [Homo sapiens] | (SEQ ID NO: 19) | | | | |
| 421 | 392.20 | 39.21 | 2 | 783.39 | NP_570843.1 | 18677767 | leucine-rich repeat protein | ENLPLGIFDHLGKLCE | 0.54 | −1.86 | Down | 5.48E−03 |
| | | | | | | | induced by beta amyloid | (SEQ ID NO: 20) | | | | |
| | | | | | | | [Homo sapiens] | | | | | |
| 254 | 360.17 | 26.45 | 3 | 1078.49 | NP_000286.2 | 21361198 | serine (or cysteine) proteinase | FLENEDRR | 0.59 | −1.71 | Down | 1.33E−03 |
| | | | | | | | inhibitor, clade A (alpha-1 | (SEQ ID NO: 21) | | | | |
| | | | | | | | antiproteinase, antitrypsin), | | | | | |
| | | | | | | | member 1; | | | | | |
| 1830 | 686.44 | 35.28 | 1 | 686.44 | NP_000286.2 | 21361198 | serine (or cysteine) proteinase | IVDLVK | 0.72 | −1.38 | Down | 6.79E−03 |
| | | | | | | | inhibitor, clade A (alpha-1 | (SEQ ID NO: 22) | | | | |
| | | | | | | | antiproteinase, antitrypsin), | | | | | |
| | | | | | | | member 1; Protease inhibitor | | | | | |
| | | | | | | | (alpha-1-antitrypsin); protease | | | | | |
| | | | | | | | inhibitor 1 (anti-elastase), | | | | | |
| | | | | | | | alpha-1-antitrypsin [Homo | | | | | |
| | | | | | | | sapiens] | | | | | |
| 626 | 426.74 | 32.88 | 2 | 852.47 | NP_000286.2 | 21361198 | serine (or cysteine) proteinase | SASLHLPK | 0.70 | −1.42 | Down | 7.70E−03 |
| | | | | | | | inhibitor, clade A (alpha-1 | (SEQ ID NO: 23) | | | | |
| | | | | | | | antiproteinase, antitrypsin), | | | | | |
| | | | | | | | member 1; Protease inhibitor | | | | | |
| | | | | | | | (alpha-1-antitrypsin); protease | | | | | |
| | | | | | | | inhibitor 1 (anti-elastase), | | | | | |
| | | | | | | | alpha-1-antitrypsin [Homo | | | | | |
| | | | | | | | sapiens] | | | | | |
| 143 | 339.20 | 54.94 | 3 | 1015.58 | NP_000286.2 | 21361198 | serine (or cysteine) proteinase | SVLGQLGITK | 0.73 | −1.36 | Down | 8.13E−03 |
| | | | | | | | inhibitor, clade A (alpha-1 | (SEQ ID NO: 24) | | | | |
| | | | | | | | antiproteinase, antitrypsin), | | | | | |
| | | | | | | | member 1; Protease inhibitor | | | | | |
| | | | | | | | (alpha-1-antitrypsin); protease | | | | | |
| | | | | | | | inhibitor 1 (anti-elastase), | | | | | |
| | | | | | | | alpha-1-antitrypsin [Homo | | | | | |
| | | | | | | | sapiens] | | | | | |
| 70 | 319.66 | 37.81 | 4 | 1275.62 | NP_000286.2 | 21361198 | serine (or cysteine) proteinase | GKWERPFEVK | 0.70 | −1.43 | Down | 9.59E−03 |
| | | | | | | | inhibitor, clade A (alpha-1 | (SEQ ID NO: 25) | | | | |
| | | | | | | | antiproteinase, antitrypsin), | | | | | |
| | | | | | | | member 1; Protease inhibitor | | | | | |
| | | | | | | | (alpha-1-antitrypsin); protease | | | | | |
| | | | | | | | inhibitor 1 (anti-elastase), | | | | | |
| | | | | | | | alpha-1-antitrypsin [Homo | | | | | |
| | | | | | | | sapiens] | | | | | |
| 2306 | 336.82 | 31.71 | 3 | 1008.44 | NP_000286.2 | 21361198 | serine (or cysteine) proteinase | QINDYVEK | 0.74 | −1.34 | Down | 9.62E−03 |
| | | | | | | | inhibitor, clade A (alpha-1 | (SEQ ID NO: 26) | | | | |
| | | | | | | | antiproteinase, antitrypsin), | | | | | |
| | | | | | | | member 1; | | | | | |

### Conclusions

The experiments described above demonstrate that the method described herein successfully tracks thousands of (de-isotoped) molecular ions for quantitative differential expression measurements using normalized raw intensities. Quantification of 4000 molecular ions in CSF and ˜7000 molecular ions in serum by LC-MS was achieved and several hundred proteins were profiled. These experiments demonstrate that isotopic labeling is not required for quantitative proteomics. The experiments above have identified differences in the expression levels of several CSF and serum proteins in multiple sclerosis that are signature biomarkers for multiple sclerosis (MS). These biomarkers can be used in a variety of diagnostic and prognostic methods, and as targets for the development of novel therapeutic strategies for the treatment of MS. This methodology is applicable to any biological fluid or tissue.

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following claims.

1. A method to identify a compound that regulates the expression or biological activity of a polypeptide or gene encoding the polypeptide, wherein the polypeptide is differentially expressed in patients with multiple sclerosis (MS), comprising:
a) contacting a test compound with a biomarker, wherein the biomarker is a polypeptide, a polynucleotide encoding the polypeptide, or a portion thereof, and wherein the expression or activity of the biomarker has been associated with MS as measured by either upregulation or downregulation of the biomarker expression or activity in serum or cerebrospinal fluid from patients with MS as compared to the level of expression or activity of the biomarker in serum or cerebrospinal fluid from non-MS controls; and
b) identifying compounds that regulate the expression or activity of the biomarker.

2. The method of claim 1, wherein step (b) comprises identifying compounds that:
i) increase the expression or activity of the biomarker if the expression of the biomarker is downregulated in the serum or cerebrospinal fluid of patients with MS as compared to the expression or activity of the biomarker in the serum or cerebrospinal fluid of non-MS controls; or
ii) decrease the expression or activity of the biomarker if the expression of the biomarker is upregulated in the serum or cerebrospinal fluid of patients with MS as compared to the expression or activity of the biomarker in the serum or cerebrospinal fluid of non-MS controls.

3. The method of claim 1, wherein the biomarker is a polynucleotide expressed by a test cell, and wherein step (b) comprises identifying compounds that regulate the expression of the polynucleotide in the presence of the test compound as compared to in the absence of the test compound.

4. The method of claim 3, wherein expression of the polynucleotide is measured by measuring transcription of the polynucleotide or translation of a protein encoded by the polynucleotide.

5. The method of claim 1, wherein the biomarker is a polypeptide, and wherein step (b) comprises identifying compounds that regulate the activity of the polypeptide in the presence of the test compound as compared to in the absence of the test compound.

6. The method of claim 1, wherein the biomarker is a polypeptide or a fragment of a polypeptide that is identified by liquid chromatography mass spectrophotometry as being differentially expressed in serum or cerebrospinal fluid from a subject with MS as compared to a subject that does not have MS.

7. The method of claim 1, wherein the biomarker is a polypeptide or biologically active fragment thereof comprising an amino acid sequence selected from the group consisting of SEQ ID NO:1-26.

8. The method of claim 1, wherein the biomarker is a polypeptide or biologically active fragment thereof selected from the group consisting of: antithrombin III; α-2 glycoprotein 1, zinc; transthyretin (prealbumin); NADH dehydrogenase (ubiquinone) 1 beta subcomplex, 2; neurotrimin; orosomucoid 1 precursor (α-1-acid glycoprotein-1); leucine-rich α-2-glycoprotein; leucine-rich repeat protein; and α-1-antitrypsin.

9. The method of claim 1, wherein the biomarker is a polypeptide or a biologically active fragment thereof comprising a polypeptide having a mass-to-charge value and a retention time (RT) value within 10% of the mass-to-charge value and RT value of a biomarker component selected from the group consisting of: component 3991, component 2298, component 100, component 132, component 4355, component 136, component 153, component 4024, component 3935, component 240, component 261, component 319, component 3573, component 359, component 373, component 376, component 377, component 408, component 573, component 579, component 2989, component 3110, component 4098, component 634, component 658, component 747, component 787, component 874, component 880, component 982, component 4164, component 4168, component 1041, component 4170, component 1104, component 4190, component 1166, component 1215, component 2607, component 3237, component 1329, component 1333, component 2844, component 1453, component 1519, component 1529, component 1546, component 2440, component 1687, component 1808, component 4797, component 1862, component 1895, component 2058, component 2970, and component 2259.

10. A method to diagnose multiple sclerosis (MS), comprising:
a) detecting in a sample of serum or cerebrospinal fluid from a patient to be tested the level of expression of at least one biomarker chosen from a panel of biomarkers whose expression has been associated with MS as measured by either upregulation or downregulation of biomarker expression in serum or cerebrospinal fluid from patients with MS as compared to the level of expression of the biomarkers in serum or cerebrospinal fluid from non-MS controls;
b) comparing the level of expression of the biomarker or biomarkers detected in the patient sample to a level of expression of the biomarker or biomarkers that has been associated with MS and a level of expression of the biomarker or biomarkers that has been associated with non-MS controls; and
c) diagnosing MS in the patient if the expression level of the biomarker or biomarkers in the patient sample is statistically more similar to the expression level of the biomarker or biomarkers that has been associated with MS than the expression level of the biomarker or biomarkers that has been associated with the non-MS controls.

11. The method of claim 10, wherein the panel of biomarkers in (a) is identified by liquid chromatography mass spectrophotometry as being differentially expressed in serum or cerebrospinal fluid from a subject with MS as compared to a subject that does not have MS.

12. The method of claim 10, wherein step (a) comprises detecting in the patient sample the expression of at least one polypeptide, biologically active fragment thereof, or polynucleotide encoding the polypeptide or biologically active fragment thereof, wherein the polypeptide comprises an amino acid sequence selected from the group consisting of SEQ ID NO:1-26;
wherein step (b) comprises comparing the level of expression of the polypeptide, fragment thereof or polynucleotide detected in the patient sample to a level of expression of the polypeptide, fragment thereof or polynucleotide that has been associated with MS and to a level of expression of the polypeptide, fragment thereof or polynucleotide that has been associated with non-MS controls; and
wherein step (c) comprises diagnosing MS in the patient, if the expression of the polypeptide, fragment thereof or polynucleotide in the patient sample is statistically more similar to the expression level of the polypeptide, fragment thereof or polynucleotide that has been associated with MS than with non-MS controls.

13. The method of claim 10, wherein the biomarker is a polypeptide, a fragment thereof, or a polynucleotide encoding the polypeptide or fragment thereof, and wherein the polypeptide is selected from the group consisting of: antithrombin III; α-2 glycoprotein 1, zinc; transthyretin (prealbumin); NADH dehydrogenase (ubiquinone) 1 beta subcomplex, 2; neurotrimin; orosomucoid 1 precursor (α-1-acid glycoprotein-1); leucine-rich α-2-glycoprotein; leucine-rich repeat protein; and α-1-antitrypsin.

14. The method of claim 10, wherein the biomarker is a polypeptide, a fragment thereof, or a polynucleotide encoding the polypeptide or fragment thereof, and wherein the polypeptide comprises a polypeptide having a mass-to-charge value and a retention time (RT) value within 10% of the mass-to-charge value and RT value of a biomarker component selected from the group consisting of: component 3991, component 2298, component 100, component 132, component 4355, component 136, component 153, component 4024, component 3935, component 240, component 261, component 319, component 3573, component 359, component 373, component 376, component 377, component 408, component 573, component 579, component 2989, component 3110, component 4098, component 634, component 658, component 747, component 787, component 874, component 880, component 982, component 4164, component 4168, component 1041, component 4170, component 1104, component 4190, component 1166, component 1215, component 2607, component 3237, component 1329, component 1333, component 2844, component 1453, component 1519, component 1529, component 1546, component 2440, component 1687, component 1808, component 4797, component 1862, component 1895, component 2058, component 2970, and component 2259.

15. The method of claim 10, wherein the step (a) of detecting comprises detecting expression of at least 2 biomarkers.

16. The method of claim 10, wherein the step (a) of detecting comprises detecting expression of at least 5 biomarkers.

17. The method of claim 10, wherein the step (a) of detecting comprises detecting expression of at least 10 biomarkers.

18. The method of claim 10, wherein the step (a) of detecting comprises detecting expression of polypeptides comprising each of SEQ ID NOs:1-26.

19. The method of claim 10, wherein expression of the biomarker is detected by measuring amounts of transcripts of a gene encoding a polypeptide biomarker in the patient serum or cerebrospinal fluid.

20. The method of claim 10, wherein expression of the biomarker is detected by detecting the expression of a protein.

21. The method of claim 10, wherein the biomarker is a polypeptide or a fragment thereof, and wherein the biological activity of the polypeptide is detected.

22. The method of claim 10, wherein the biomarker is a polypeptide, and wherein the expression of the polypeptide is detected using an antibody that selectively binds to the polypeptide, or an antigen binding fragment thereof.

23. The method of claim 10, further comprising determining if the patient has relapsing/remitting MS or a progressive form of MS, the step of determining comprising:
a) detecting in the sample the level of expression of at least one biomarker chosen from a panel of biomarkers whose expression has been associated with relapsing/remitting MS and or a progressive form of MS as measured by either upregulation or downregulation of biomarker expression in serum or cerebrospinal fluid from patients with relapsing/remitting MS as compared to the level of expression of the biomarkers in serum or cerebrospinal fluid from subjects with a progressive form of MS;
b) comparing the level of expression of the biomarker detected in the patient sample to a level of expression of the biomarker that has been associated with the relapsing/remitting MS and to a level of expression of the biomarker that has been associated with the progressive form of MS; and
c) diagnosing relapsing/remitting MS in the patient, if the expression of the biomarker in the patient sample is statistically more similar to the expression level of the biomarker that has been associated with relapsing/remitting MS than with the progressive form of MS, or diagnosing progressive form of MS in the patient, if the expression of the biomarker in the patient sample is statistically more similar to the expression level of the biomarker that has been associated with progressive form of MS than with relapsing/remitting MS.

24. The method of claim 10, wherein the level of expression of the biomarker that has been associated with MS and the level of expression of the biomarker that has been associated with non-MS controls has been predetermined.

25. A plurality of antibodies or antigen binding fragments thereof for the detection of the expression of proteins that are associated with multiple sclerosis (MS) in a patient, wherein the plurality of antibodies or antigen binding fragments thereof consists of at least two antibodies or antigen binding fragments thereof, each of which selectively binds to a polypeptide, the expression of which is regulated differently in serum or cerebrospinal fluid of patients with MS as compared to serum or cerebrospinal fluid of individuals that do not have MS.

26. The plurality of antibodies or antigen binding fragments thereof of claim 25, wherein each antibody or antigen binding fragment thereof selectively binds to a polypeptide comprising an amino acid sequence selected from the group consisting of SEQ ID NOs:1-26.

27. The plurality of antibodies or antigen binding fragments thereof of claim 25, wherein the plurality comprises antibodies or antigen binding fragments thereof that selectively bind to at least two polypeptides comprising an amino acid sequence selected from the group consisting of SEQ ID NOs:1-26.

28. The plurality of antibodies or antigen binding fragments thereof of claim 25, wherein each antibody or antigen binding fragment thereof selectively binds to a polypeptide selected from the group of: antithrombin III; α-2 glycoprotein 1, zinc; transthyretin (prealbumin); NADH dehydrogenase (ubiquinone) 1 beta subcomplex, 2; neurotrimin; orosomucoid 1 precursor (α-1-acid glycoprotein-1); leucine-rich α-2-glycoprotein; leucine-rich repeat protein; and α-1-antitrypsin.

29. The plurality of antibodies or antigen binding fragments thereof of claim 25, wherein each antibody or antigen binding fragment thereof selectively binds to a polypeptide comprising a polypeptide having a mass-to-charge value and a retention time (RT) value within 10% of the mass-to-charge value and RT value of a biomarker component selected from the group consisting of: component 3991, component 2298, component 100, component 132, component 4355, component 136, component 153, component 4024, component 3935, component 240, component 261, component 319, component 3573, component 359, component 373, component 376, component 377, component 408, component 573, component 579, component 2989, component 3110, component 4098, component 634, component 658, component 747, component 787, component 874, component 880, component 982, component 4164, component 4168, component 1041, component 4170, component 1104, component 4190, component 1166, component 1215, component 2607, component 3237, component 1329, component 1333, component 2844, component 1453, component 1519, component 1529, component 1546, component 2440, component 1687, component 1808, component 4797, component 1862, component 1895, component 2058, component 2970, and component 2259.

30. The plurality of antibodies or antigen binding fragments thereof of claim 25, wherein the antibodies or antigen binding fragments thereof are immobilized on a substrate.

31. The plurality of antibodies or antigen binding fragments thereof of claim 25, wherein the antibodies or antigen binding fragments thereof are conjugated to detectable markers.


SEQUENCE LISTING (26 sequences)

SEQ ID NO: 1 - protein, 13 aa, Homo sapiens
FATTFYQHLADSK

SEQ ID NO: 2 - protein, 16 aa, Homo sapiens
EIPAWVPFDPAAQITK

SEQ ID NO: 3 - protein, 13 aa, Homo sapiens
GSPAINVAVHVFR

SEQ ID NO: 4 - protein, 23 aa, Homo sapiens
ALGISPFHEHAEVVFTANDSGPR

SEQ ID NO: 5 - protein, 23 aa, Homo sapiens
ALGISPFHEHAEVVFTANDSGPR

SEQ ID NO: 6 - protein, 23 aa, Homo sapiens
ALGISPFHEHAEVVFTANDSGPR

SEQ ID NO: 7 - protein, 6 aa, Homo sapiens
VLDAVR

SEQ ID NO: 8 - protein, 13 aa, Homo sapiens
GSPAINVAVHVFR

SEQ ID NO: 9 - protein, 28 aa, Homo sapiens
TSESGELHGLTTEEEFVEGIYKVEIDTK

SEQ ID NO: 10 - protein, 16 aa, Homo sapiens
SPYSYSTTAVVTNPKE

SEQ ID NO: 11 - protein, 14 aa, Homo sapiens
GSPAINVAVHVFRK

SEQ ID NO: 12 - protein, 14 aa, Homo sapiens
GSPAINVAVHVFRK

SEQ ID NO: 13 - protein, 28 aa, Homo sapiens
TSESGELHGLTTEEEFVEGIYKVEIDTK

SEQ ID NO: 14 - protein, 24 aa, Homo sapiens
SGCARTAGDGGVRHAGGGVHIEPR

SEQ ID NO: 15 - protein, 15 aa, Homo sapiens
VKVTVNYPPYISEAK

SEQ ID NO: 16 - protein, 9 aa, Homo sapiens
SDVVYTDWK

SEQ ID NO: 17 - protein, 25 aa, Homo sapiens
HLLILRDTKTYMLAFDVNDEKNWGL

SEQ ID NO: 18 - protein, 9 aa, Homo sapiens
WFYIASAFR

SEQ ID NO: 19 - protein, 10 aa, Homo sapiens
VAAGAFQGLR

SEQ ID NO: 20 - protein, 16 aa, Homo sapiens
ENLPLGIFDHLGKLCE

SEQ ID NO: 21 - protein, 8 aa, Homo sapiens
FLENEDRR

SEQ ID NO: 22 - protein, 6 aa, Homo sapiens
IVDLVK

SEQ ID NO: 23 - protein, 8 aa, Homo sapiens
SASLHLPK

SEQ ID NO: 24 - protein, 10 aa, Homo sapiens
SVLGQLGITK

SEQ ID NO: 25 - protein, 10 aa, Homo sapiens
GKWERPFEVK

SEQ ID NO: 26 - protein, 8 aa, Homo sapiens
QINDYVEK